SEQUENCE LISTING (15 sequences)

SEQ ID NO: 1 - DNA, 864 bp, Akkermansia muciniphila
  CDS (1)..(864)
atcgtcaattccaaacgcagtgaactggacaaaaaaatcagcatcgccgccaaggaaatcaagtccgccaatgctgcggaaatcactccgagccgatcatccaacgaagagctggaaaaagaactgaaccgctatgccaaggccgtgggcagcctggaaacggcctacaagcccttccttgcctcctccgcgctggtccccaccacgcccacggcattccagaatgaactgaaaacattcagggattccctgatctcctcctgcaagaaaaagaacattctcataacggacacatcctcctggctcggtttccaggtttacagcacccaggctccctctgttcaggcggcctccacgctgggttttgaattgaaagccatcaacagcctggtcaacaaactggcggaatgcggcctgtccaaattcatcaaggtgtaccgcccccagctccccattgaaaccccggcgaacaatccggaagaatcggacgaagccgaccaggccccatggactcccatgcctctggaaatagccttccagggcgaccgggaaagtgtattgaaagccatgaacgccataaccggcatgcaggactatctgttcacggtcaactccatccgtatccgcaacgaacggatgatgccccctcccatcgccaatccggcagccgccaaacctgccgcggcccaacccgccacgggtgcggcttccctgactccggcggatgaggcggctgcacctgcagccccggccatccagcaagtcatcaagccttacatgggcaaggagcaggtctttgtccaggtctccctgaatctggtccacttcaaccagcccaaggctcaggaaccgtctgaagattaa

SEQ ID NO: 2 - protein, 287 aa, Akkermansia muciniphila
IVNSKRSELDKKISIAAKEIKSANAAEITPSRSSNEELEKELNRYAKAVGSLETAYKPFLASSALVPTTPTAFQNELKTFRDSLISSCKKKNILITDTSSWLGFQVYSTQAPSVQAASTLGFELKAINSLVNKLAECGLSKFIKVYRPQLPIETPANNPEESDEADQAPWTPMPLEIAFQGDRESVLKAMNAITGMQDYLFTVNSIRIRNERMMPPPIANPAAAKPAAAQPATGAASLTPADEAAAPAAPAIQQVIKPYMGKEQVFVQVSLNLVHFNQPKAQEPSED

SEQ ID NO: 3 - DNA, 864 bp, Artificial
  Amuc_1100 optimized for Bifidobacterium
attgtgaactccaagcgctccgagctggacaagaagatcagcattgccgctaaggagatcaagtccgccaatgctgccgagatcacgccctccaggagcagcaacgaggagctggaaaaggagctgaaccggtatgccaaagcggtgggtagcctggaaaccgcgtacaaacccttccttgcgtcctcggcgctcgttccgaccaccccgacggccttccagaacgagctcaagacgttccgcgactccctcatctcgtcctgcaagaagaagaacatcctcatcaccgatacgagctcctggttgggcttccaggtgtactccacccaggccccgtcggtccaagccgcctcgaccttgggcttcgaactgaaggccatcaactccctggtgaacaagctggccgaatgcgggctgtccaagttcatcaaggtgtatcgtccgcagctccccatcgaaaccccggccaacaaccccgaggaatccgacgaggccgatcaggcgccctggaccccgatgcctctcgagatcgcctttcagggcgatcgcgagtccgtgctgaaggcgatgaacgccatcaccggcatgcaggactaccttttcacggtgaacagcatccgcatccggaacgagcgcatgatgccgccgccgattgcgaatccggcggccgcgaaaccggcagctgcccaaccggccactggagcagccagcctgacccctgcggacgaggcagccgctcctgcagctccggcgatccaacaggtcatcaagccgtacatgggcaaggaacaggtgttcgtccaggtttccctgaacctggtccacttcaaccagcccaaagcccaggaaccgtcggaggactga

SEQ ID NO: 4 - DNA, 864 bp, Artificial
  Amuc_1100 optimized for Bacillus sequence
attgtgaactcaaaacggtctgagttggacaagaaaatcagcatagctgcaaaagagatcaaatccgcaaacgcagcagaaattacgccgtcaagaagttccaacgaagagctggagaaagaactgaatcgctatgccaaagcggttggatcacttgaaacggcatacaagccgtttcttgcgagctctgcccttgtaccgacaacaccgacagcgttccaaaacgaactgaaaacatttcgtgacagccttatatcttcctgcaagaagaagaacatcctcatcactgatacaagctcttggttaggctttcaggtgtatagcacacaagcaccttcagttcaagcggcatcaacgttaggctttgagctgaaagccatcaattcgttggtgaacaaacttgcggaatgtggcttatcgaagtttatcaaagtctatcgtccgcaattacccattgaaaccccagcaaataaccctgaagaatcggatgaggcggatcaagccccttggaccccaatgcctttggaaattgcctttcagggtgatagagaatctgttttaaaagccatgaatgcgattaccggaatgcaggactatctgttcacggtcaatagtattcgcattcgaaatgagaggatgatgccaccgccgattgctaatcctgcagccgctaaaccagctgctgctcaaccggcaactggagctgcaagtctgactcctgcggatgaagcggctgctccagctgcccctgcgattcaacaggtaatcaaaccgtacatggggaaagaacaggtatttgtccaggtttcattgaatctcgtgcatttcaatcagccgaaagcccaagaacccagcgaagattaa

SEQ ID NO: 5 - DNA, 864 bp, Artificial
  Amuc_1100 optimized for Brevibacillus species
atcgtcaatagcaaacgcagtgaactggacaagaaaatctccattgccgcaaaagagatcaaatccgcaaacgctgccgaaatcactccctctcgtagttctaacgaggaactggagaaagaactgaatcgctatgctaaagccgtaggctctctggaaaccgcgtacaaaccgtttcttgcgtcctctgcattggtccccaccacaccgaccgcgtttcagaatgagctgaaaaccttccgcgattctctgatctcgagctgcaagaagaagaacatcctcatcaccgacacatcgtcctggttgggattccaagtatactccacgcaagctccaagcgtacaagcggcatcgactcttggctttgagctgaaagctatcaactccctcgttaacaagctcgcggagtgtggcctttccaaattcatcaaggtgtatcgacctcagctgccaatcgaaactccggctaacaaccctgaagaatccgatgaagcagatcaagccccatggactccgatgccactggaaatcgcgtttcaaggtgaccgtgaatccgtactgaaagccatgaacgcaatcacggggatgcaagactacttgttcacggtgaactccattcgcattcgcaatgaacgcatgatgccacctccaattgcgaatcctgcagctgcaaaaccagctgcggcacaacccgctacaggtgcggcatccttgactccggcagacgaagctgctgctccagctgcgcctgcaatccagcaagtgatcaaaccctatatgggcaaagaacaggttttcgtacaggtttccctgaatctggtgcatttcaaccaaccgaaagcgcaagaaccttccgaagattaa

SEQ ID NO: 6 - DNA, 864 bp, Artificial
  Amuc_1100 optimized for Lactococcus species
atagttaacagcaaacgatcagagttagacaagaaaatttcaattgcagcaaaggagataaaatctgccaatgctgctgagattactccctctagaagttcaaacgaagaacttgagaaagaattgaatagatatgcgaaagcggttggttcacttgaaaccgcgtataaaccgtttctagcgagttctgccttagtaccaactacaccaacggcatttcagaatgaacttaaaacttttagagacagcttaatttcatcatgcaagaagaagaacatacttattacagatacctcatcatggttaggatttcaggtttatagtactcaagctccttcagttcaagccgcatcaacgttgggttttgagttgaaagcgattaatagcttagtaaacaaacttgctgaatgtgggttgagtaaatttatcaaagtctatagaccgcaattacctattgaaactcccgctaataatccagaagaaagtgatgaagcagatcaagcaccatggacacctatgcctttggaaattgcctttcaaggagatcgagaaagtgttttaaaagccatgaatgcaattacaggaatgcaagattacttattcaccgtcaattctattcgtatccgtaatgaacgcatgatgcctccacctattgcaaatcctgcagctgctaaaccggctgcagcacaaccagctacaggtgcagcttctctaacaccagccgatgaagctgctgctccagctgcaccagccatacaacaggtaatcaaaccttatatgggcaaagaacaagtgtttgttcaagtgtctttaaatttagttcatttcaatcaaccaaaagctcaagaaccatcagaagattaa

SEQ ID NO: 7 - DNA, 864 bp, Artificial
  Amuc_1100 optimized for Saccharomyces species
attgttaattctaagagatccgaactggacaagaaaatctcgattgcagcgaaggaaatcaaatcggctaatgcagctgaaatcactccttcaaggtctagtaacgaggaattggagaaagaattgaacagatatgctaaagcagttggtagcttggaaacagcctataaaccgttcttagcatctagcgcattagttccaaccactccaacagcgtttcagaatgaactgaaaacgtttagagacagcttgattagttcttgcaagaagaagaacatcttgataacagacaccagttcatggttaggctttcaagtatactctactcaagcaccatcagttcaagctgcatccactttgggattcgagttaaaggccataaactcacttgtgaacaaacttgctgaatgtggtctatccaagttcatcaaagtttacagaccccagttaccgattgaaactcccgcaaataatcctgaagagtcagatgaagccgatcaagctccttggacacctatgcctctagaaattgcttttcagggtgatagagagagtgtattgaaagcgatgaatgccattacaggtatgcaagattacctatttaccgtaaattccattaggatacgtaacgagagaatgatgccaccaccaattgccaatcctgctgcagccaaacccgctgccgctcaaccagcgactggagcagcatctcttacgccagccgatgaagctgcagctccagctgctcctgccatacaacaggtgataaaaccctatatggggaaagaacaggtctttgtccaagtctcgttgaatttagtgcatttcaaccaaccaaaggctcaagaaccgtctgaggattaa

SEQ ID NO: 8 - DNA, 1080 bp, Artificial
  Alanine racemase (alr)
atgcaagcggcaactgttgtgattaaccgccgcgctctgcgacacaacctgcaacgtcttcgtgaactggcccctgccagtaaaatggttgcggtggtgaaagcgaacgcttatggtcacggtcttcttgagaccgcgcgaacgctccccgatgctgacgcctttggcgtagcccgtctcgaagaagctctgcgactgcgtgcggggggaatcaccaaacctgtactgttactcgaaggcttttttgatgccagagatctgccgacgatttctgcgcaacattttcataccgccgtgcataacgaagaacagctggctgcgctggaagaggctagcctggacgagccggttaccgtctggatgaaactcgataccggtatgcaccgtctgggcgtaaggccggaacaggctgaggcgttttatcatcgcctgacccagtgcaaaaacgttcgtcagccggtgaatatcgtcagccattttgcgcgcgcggatgaaccaaaatgtggcgcaaccgagaaacaactcgctatctttaataccttttgcgaaggcaaacctggtcaacgttccattgccgcgtcgggtggcattctgctgtggccacagtcgcattttgactgggtgcgcccgggcatcattctttatggcgtctcgccgctggaagatcgctccaccggtgccgattttggctgtcagccagtgatgtcactaacctccagcctgattgccgtgcgtgagcataaagccggagagcctgttggttatggtggaacctgggtaagcgaacgtgatacccgtcttggcgtagtcgcgatgggctatggcgatggttatccgcgcgccgcgccgtccggtacgccagtgctggtgaacggtcgcgaagtaccgattgtcgggcgcgtggcgatggatatgatctgcgtagacttaggtccacaggcgcaggacaaagccggggatccggtcattttatggggcgaaggtttgcccgtagaacgtatcgctgaaatgacgaaagtaagcgcttacgaacttattacgcgcctgacttcaagggtcgcgatgaaatacgtggattaa

SEQ ID NO: 9 - DNA, 6787 bp, Artificial
  p3050Alr_Amuc1100_sh71
atatgaaaaaatttaactttaaaaccatgttgctattagttttggctagttgtgtcttcggggtcgtcgttaacgtgactactagtcttggaccacaaaccgcaatcaccgcccaggcctccaaggtcgacatcgtcaattccaaacgcagtgaactggacaaaaaaatcagcatcgccgccaaggaaatcaagtccgccaatgctgcggaaatcactccgagccgatcatccaacgaagagctggaaaaagaactgaaccgctatgccaaggccgtgggcagcctggaaacggcctacaagcccttccttgcctcctccgcgctggtccccaccacgcccacggcattccagaatgaactgaaaacattcagggattccctgatctcctcctgcaagaaaaagaacattctcataacggacacatcctcctggctcggtttccaggtttacagcacccaggctccctctgttcaggcggcctccacgctgggttttgaattgaaagccatcaacagcctggtcaacaaactggcggaatgcggcctgtccaaattcatcaaggtgtaccgcccccagctccccattgaaaccccggcgaacaatccggaagaatcggacgaagccgaccaggccccatggactcccatgcctctggaaatagccttccagggcgaccgggaaagtgtattgaaagccatgaacgccataaccggcatgcaggactatctgttcacggtcaactccatccgtatccgcaacgaacggatgatgccccctcccatcgccaatccggcagccgccaaacctgccgcggcccaacccgccacgggtgcggcttccctgactccggcggatgaggcggctgcacctgcagccccggccatccagcaagtcatcaagccttacatgggcaaggagcaggtctttgtccaggtctccctgaatctggtccacttcaaccagcccaaggctcaggaaccgtctgaagattaaaagcttcaaattacagcacgtgttgctttgattgatagccaaaaagcagcagttgataaagcaattactgatattgctgaaaaattgtaatttataaataaaaatcaccttttagaggtggtttttttatttataaattattcgtttgatttcgctttcgatagaacaatcaaagcgagaataaggaagataaatcccataagggcgggagcagaatgtccgagactaattcatggatcgattttttattaaaacgtctcaaaatcgtttctgagacgttttagcgtttatttcgtttagttatcggcataatcgttaaaacaggcgttatcgtagcgtaaaagcccttgagcgtagcgtgctttgcagcgaagatgttgtctgttagattatgaaagccgatgactgaatgaaataataagcgcagcgtccttctatttcggttggaggaggctcaagggagtttgagggaatgaaattccctcatgggtttgattttaaaaattgcttgcaattttgccgagcggtagcgctggaaaaatttttgaaaaaaatttggaatttggaaaaaaatggggggaaaggaagcgaattttgcttccgtactacgaccccccattaagtgccgagtgccaatttttgtgccaaaaacgctctatcccaactggctcaagggtttgaggggtttttcaatcgccaacgaatcgccaacgttttcgccaacgttttttataaatctatatttaagtagctttattgttgtttttatgattacaaagtgatacactaattttataaaattatttgattggagttttttaaatggtgatttcagaatcgaaaaaaagagttatgatttctctgacaaaagagcaagataaaaaattaacagatatggcgaaacaaaaaggtttttcaaaatctgcggttgcggcgttagctatagaagaatatgcaagaaaggaatcagaataaaaaaaataagcgaaagctcgcgtttttagaaggatacgagttttcgctacttgtttttgataaggtaatatatcatggctattaaatactaaagctagaaattttggatttttattatatcctgactcaattcctaatgattggaaagaaaaattagagagtttgggcgtatctatggctgtcagtcctttacacgatatggacgaaaaaaaagataaagatacatggaatagtagtgatgttatacgaaatggaaagcactataaaaaaccacactatcacgttatatatattgcacgaaatcctgtaacaatagaaagcgttaggaacaagattaagcgaaaattggggaatagttcagttgctcatgttgagatacttgattatatcaaaggttcatatgaatatttgactcatgaatcaaaggacgctattgctaagaataaacatatatacgacaaaaaagatattttgaacattaatgattttgatattgaccgctatataacacttgatgaaagccaaaaaagagaattgaagaatttacttttagatatagtggatgactataatttggtaaatacaaaagatttaatggcttttattcgccttaggggagcggagtttggaattttaaatacgaatgatgtaaaagatattgtttcaacaaactctagcgcctttagattatggtttgagggcaattatcagtgtggatatagagcaagttatgcaaaggttcttgatgctgaaacgggggaaataaaatgacaaacaaagaaaaagagttatttgctgaaaatgaggaattaaaaaaagaaattaaggacttaaaagagcgtattgaaagatacagagaaatggaagttgaattaagtacaacaatagatttattgagaggagggattattgaataaataaaagcccccctgacgaaagtcgaagggggcttttattttggtttgatgttgcgattaatagcaatacgattgcaataaacaaaaggatccatgcaagcggcaactgttgtgattaaccgccgcgctctgcgacacaacctgcaacgtcttcgtgaactggcccctgccagtaaaatggttgcggtggtgaaagcgaacgcttatggtcacggtcttcttgagaccgcgcgaacgctccccgatgctgacgcctttggcgtagcccgtctcgaagaagctctgcgactgcgtgcggggggaatcaccaaacctgtactgttactcgaaggcttttttgatgccagagatctgccgacgatttctgcgcaacattttcataccgccgtgcataacgaagaacagctggctgcgctggaagaggctagcctggacgagccggttaccgtctggatgaaactcgataccggtatgcaccgtctgggcgtaaggccggaacaggctgaggcgttttatcatcgcctgacccagtgcaaaaacgttcgtcagccggtgaatatcgtcagccattttgcgcgcgcggatgaaccaaaatgtggcgcaaccgagaaacaactcgctatctttaataccttttgcgaaggcaaacctggtcaacgttccattgccgcgtcgggtggcattctgctgtggccacagtcgcattttgactgggtgcgcccgggcatcattctttatggcgtctcgccgctggaagatcgctccaccggtgccgattttggctgtcagccagtgatgtcactaacctccagcctgattgccgtgcgtgagcataaagccggagagcctgttggttatggtggaacctgggtaagcgaacgtgatacccgtcttggcgtagtcgcgatgggctatggcgatggttatccgcgcgccgcgccgtccggtacgccagtgctggtgaacggtcgcgaagtaccgattgtcgggcgcgtggcgatggatatgatctgcgtagacttaggtccacaggcgcaggacaaagccggggatccggtcattttatggggcgaaggtttgcccgtagaacgtatcgctgaaatgacgaaagtaagcgcttacgaacttattacgcgcctgacttcaagggtcgcgatgaaatacgtggattaaacacgttactaaagggaatggagaccggggcccttcaatagagttcttaacgttaatccgaaaaaaactaacgttaatattaaaaaataagatccgcttgtgaattatgtataatttgattagactaaagaataggagaaagtatgatgatatttaaaaaactttctcgttaagataggttgttggtgagcatgttatatacggatgtatcggtttccttaatgcaaaattttgttgctatcttattaatttttctattatatagatatattcaaagaaagataacatttaaacggatcatattagatattttaatagcgattattttttcaatattatatctgtttatttcagatgcgtcattacttgtaatggtattaatgcgattagggtggcattttcatcaacaaaaagaaaataagataaaaacgactgatacagctaatttaattctaattatcgtgatccagttattgttagttgcggttgggactattattagtcagtttaccatatcgattatcaaaagtgatttcagccaaaatatattgaacaatagtgcaacagatataactttattaggtattttctttgctgttttatttgacggcttgttctttatattattgaagaataagcggactgaattacaacatttaaatcaagaaatcattgaattttcgttagaaaaacaatattttatatttatatttattttatttatagtaatagaaattattttagcagttgggaatcttcaaggagtaacagccacgatattattaaccattatcattattttttgtgtccttatcgggatgactttttggcaagtgatgctttttttgaaggcttattcgattcgccaagaagccaatgaccaattggtccggaatcaacaacttcaagattatctagtcaatatcgaacagcagtacaccgaattacggcgatttaagcatgattatcaaaacatcttattatcgttggagagttttgccgaaaagggcgatcagcaacagtttaaggcgtattaccaagaattattagcacaacggccaattcaaagtgaaatccaaggggcagtcattgcacaactcgactacttgaaaaatgatcctattcgaggattagtcattcaaaagtttttggcagccaaacaggctggtgttactttaaaattcgaaatgaccgaaccaatcgaattagcaaccgctaatctattaacggttattcggattatcggtattttattagacaatgcgattgaacaagccgttcaagaaaccgatcaattggtgagttgtgctttcttacaatctgatggtttaatcgaaattacgattgaaaatacggccagtcaagttaagaatctccaagcattttcagagttaggctattcaacgaaaggcgctggtcgggggactggtttagctaatgtgcaggatttgattgccaaacaaaccaatttattcttagaaacacagattgaaaatagaaagttacgacagacattgatgattacggaggaaacttaatttgtatcccgtttatttattagaggatgatttacagcaacaagcgatttatcagcaaattatcgcgaatacgattatgattaacgaatttgcaatgactttaacatgcgctgccagtgatactgagacattgttggcggcaattaaggatcagcaacgaggtttattctttttggatatggaaattgaggataaccgccaagccggtttagaagtggcaactaagattcggcagatgatgccgtttgcgcaaattgtcttcattacaacccacgaggaactgacattattaacgttagaacgaaaaatagcgcctttagattacattctcaaggaccaaacaatggctgaaatcaaaaggcaattgattgatgatctattgttagctgagaagcaaaacgaggcggcagcgtatcaccgagaaaatttatttagttataaaataggtcctcgctttttctcattaccattaaaggaagttgtttatttatatactgaaaaagaaaatccgggtcatattaatttgttagccgttaccagaaaggttacttttccaggaaatttaaatgcgctggaagcccaatatccaatgctctttcggtgtgataaaagttacttagttaacctatctaatattgccaattatgacagtaaaacacggagtttaaaatttgtagatggcagtgaggcaaaagtctcgttccggaaatcacgggaactagtggccaaattaaaacaaatgatgtagcgcctgcagcacgccaaatgatcccagtaaaaagccacccgcatggcgggtggctttttattagccctagaagggcttcccacacgcatttcagcgccttagtgccttagtttgtgaatcataggtggtatagtcccgaaatacccgtctaaggaattgtcagataggcctaatgactggcttttataatatgagataatgccgactgtactttttacagtcggttttctaatgtcactaacctgccccgttagttgaagaaggtttttatattacagctccagatctaccggtgggcccatattaacgtttaaccgataaagttgaacgttaatattttttttgcgcagaaatggtaaattgaagcataatagtcttgtaaggtatttagctggctggcgtaaagtatgctttataaaataatatataggagtatgattc

SEQ ID NO: 10 - protein, 517 aa, Homo sapiens
MLRFLAPRLLSLQGRTARYSSAAALPSPILNPDIPYNQLFINNEWQDAVSKKTFPTVNPTTGEVIGHVAEGDRADVDRAVKAAREAFRLGSPWRRMDASERGRLLNLLADLVERDRVYLASLETLDNGKPFQESYALDLDEVIKVYRYFAGWADKWHGKTIPMDGQHFCFTRHEPVGVCGQIIPWNFPLVMQGWKLAPALATGNTVVMKVAEQTPLSALYLASLIKEAGFPPGVVNIITGYGPTAGAAIAQHVDVDKVAFTGSTEVGHLIQKAAGDSNLKRVTLELGGKSPSIVLADADMEHAVEQCHEALFFNMGQCCCAGSRTFVEESIYNEFLERTVEKAKQRKVGNPFELDTQQGPQVDKEQFERVLGYIQLGQKEGAKLLCGGERFGERGFFIKPTVFGGVQDDMRIAKEEIFGPVQPLFKFKKIEEVVERANNTRYGLAAAVFTRDLDKAMYFTQALQAGTVWVNTYNIVTCHTPFGGFKESGNGRELGEDGLKAYTEVKTVTIKVPQKNS

SEQ ID NO: 11 - DNA, 8577 bp, Artificial
  p3050alarAmuc_1100_alcA-al1b1-sh71
  misc_feature (2)..(124): 3050
  5′UTR (125)..(131)
  gene (132)..(995): Amuc_1100
  3′UTR (996)..(1003)
  terminator (1004)..(1042): Terminator BBa_B1006
  promoter (1043)..(1408): alcA promoter GB KC189907.1
  5′UTR (1409)..(1415)
  misc_feature (1416)..(2969): AL1B1_HUMAN
  3′UTR (2970)..(2977)
  terminator (2978)..(3016): Terminator BBa_B1006
  rep_origin (3017)..(4763): sh71rep
  misc_feature (4771)..(5850): alr
  misc_feature (6042)..(7387): sppK
  misc_feature (7390)..(8132): sppR
atatgaaaaaatttaactttaaaaccatgttgctattagttttggctagttgtgtcttcggggtcgtcgttaacgtgactactagtcttggaccacaaaccgcaatcaccgcccaggcctccaaaggaggtatcgtcaattccaaacgcagtgaactggacaaaaaaatcagcatcgccgccaaggaaatcaagtccgccaatgctgcggaaatcactccgagccgatcatccaacgaagagctggaaaaagaactgaaccgctatgccaaggccgtgggcagcctggaaacggcctacaagcccttccttgcctcctccgcgctggtccccaccacgcccacggcattccagaatgaactgaaaacattcagggattccctgatctcctcctgcaagaaaaagaacattctcataacggacacatcctcctggctcggtttccaggtttacagcacccaggctccctctgttcaggcggcctccacgctgggttttgaattgaaagccatcaacagcctggtcaacaaactggcggaatgcggcctgtccaaattcatcaaggtgtaccgcccccagctccccattgaaaccccggcgaacaatccggaagaatcggacgaagccgaccaggccccatggactcccatgcctctggaaatagccttccagggcgaccgggaaagtgtattgaaagccatgaacgccataaccggcatgcaggactatctgttcacggtcaactccatccgtatccgcaacgaacggatgatgccccctcccatcgccaatccggcagccgccaaacctgccgcggcccaacccgccacgggtgcggcttccctgactccggcggatgaggcggctgcacctgcagccccggccatccagcaagtcatcaagccttacatgggcaaggagcaggtctttgtccaggtctccctgaatctggtccacttcaaccagcccaaggctcaggaaccgtctgaagattaatacttgaaaaaaaaaaaccccgcccctgacagggcggggtttttttttccattgtggtgatcgttccgacatgcttgtctgcatgggtttctgcgtgtcgggactcaagtgatctggggcttgatgcatgtgggacagcacgaggtagaggtggaaactgacatacgactccgttacatgccccgtttaagcgctatgcgtatcgtgccgtctaatcccgtgatggagcgttatcaggcacagtacggactggatgccctcatggcgaaccacaaacctcaggagctccctacgtactgagctatccgcgcattgcttcgcctcatagctaaacgggcatgacacacaatccgaccatactcaggaaaacgcttccactgtacaaagaggtccacttcatctggagaggccctaggaggtatgctcagattcttggcgcctcgccttcttagcctccaaggacgtacagccagatattcaagtgcagcagctcttccgagcccgattctcaatccggatattccgtataaccaactgttcattaacaacgagtggcaagacgcagtaagcaagaaaacgtttccgacagtcaatccaactaccggagaagtgatcggccacgttgcagaaggtgatcgggccgatgtcgatcgtgcagttaaagctgcgagagaggctttcaggcttgggtccccatggcggaggatggatgcttcggaacgtggcagactgctcaatctgttagctgatcttgtagagcgagatcgggtatatctggcatctctggaaacactggacaatgggaagccatttcaggaatcctatgcccttgatctggatgaggtgattaaggtgtatcgctattttgctggctgggcagataagtggcatgggaaaacaataccgatggacggccagcacttttgctttaccagacatgaacctgttggagtatgtggtcaaatcataccctggaactttccgctggtaatgcaaggctggaaattagcacccgcgttagcgacgggtaatacagtggtcatgaaagtagctgagcaaacgccgctttcagccttgtatttagcctctcttatcaaagaagctggatttcctccgggtgttgttaacatcattacaggatacggccctacagctggcgcggcaatcgcgcaacatgtggacgtagacaaagtcgcctttactggctcaaccgaagtcgggcatctgatccagaaagctgctggcgatagcaacttgaaacgcgttacactggagttaggaggaaaatctccgagtattgtcttagcggatgcagatatggaacatgctgttgaacagtgccatgaagccttattcttcaacatgggtcagtgctgttgtgcgggatctcgtacctttgtggaagagtccatttacaatgaatttctggaacgtaccgttgagaaggcgaaacaacgcaaagtcggaaatccgtttgagctggacacgcaacaaggtccacaagtggacaaagaacagtttgaaagagttttgggctacattcagctcggacagaaagaaggagccaagttactttgcggaggcgaacgatttggtgaacggggtttcttcatcaaaccaactgtctttggtggagtgcaggatgacatgaggattgcgaaagaagagattttcggccctgtgcaacctctgttcaaatttaagaaaatcgaagaagttgtggaaagagccaacaatacgcggtatggccttgcggcggcagtctttactcgcgatttagacaaggcgatgtactttacgcaagccttgcaggcagggacagtttgggtgaatacgtataacattgttacatgtcacacaccttttggaggctttaaagagtcagggaatggacgagaattgggcgaagatgggttgaaagcatacactgaggtcaaaacagtcacgataaaagtaccccagaagaattcgtaatacttgaaaaaaaaaaaccccgcccctgacagggcggggttttttttcatggatcgattttttattaaaacgtctcaaaatcgtttctgagacgttttagcgtttatttcgtttagttatcggcataatcgttaaaacaggcgttatcgtagcgtaaaagcccttgagcgtagcgtgctttgcagcgaagatgttgtctgttagattatgaaagccgatgactgaatgaaataataagcgcagcgtccttctatttcggttggaggaggctcaagggagtttgagggaatgaaattccctcatgggtttgattttaaaaattgcttgcaattttgccgagcggtagcgctggaaaaatttttgaaaaaaatttggaatttggaaaaaaatggggggaaaggaagcgaattttgcttccgtactacgaccccccattaagtgccgagtgccaatttttgtgccaaaaacgctctatcccaactggctcaagggtttgaggggtttttcaatcgccaacgaatcgccaacgttttcgccaacgttttttataaatctatatttaagtagctttattgttgtttttatgattacaaagtgatacactaattttataaaattatttgattggagttttttaaatggtgatttcagaatcgaaaaaaagagttatgatttctctgacaaaagagcaagataaaaaattaacagatatggcgaaacaaaaaggtttttcaaaatctgcggttgcggcgttagctatagaagaatatgcaagaaaggaatcagaataaaaaaaataagcgaaagctcgcgtttttagaaggatacgagttttcgctacttgtttttgataaggtaatatatcatggctattaaatactaaagctagaaattttggatttttattatatcctgactcaattcctaatgattggaaagaaaaattagagagtttgggcgtatctatggctgtcagtcctttacacgatatggacgaaaaaaaagataaagatacatggaatagtagtgatgttatacgaaatggaaagcactataaaaaaccacactatcacgttatatatattgcacgaaatcctgtaacaatagaaagcgttaggaacaagattaagcgaaaattggggaatagttcagttgctcatgttgagatacttgattatatcaaaggttcatatgaatatttgactcatgaatcaaaggacgctattgctaagaataaacatatatacgacaaaaaagatattttgaacattaatgattttgatattgaccgctatataacacttgatgaaagccaaaaaagagaattgaagaatttacttttagatatagtggatgactataatttggtaaatacaaaagatttaatggcttttattcgccttaggggagcggagtttggaattttaaatacgaatgatgtaaaagatattgtttcaacaaactctagcgcctttagattatggtttgagggcaattatcagtgtggatatagagcaagttatgcaaaggttcttgatgctgaaacgggggaaataaaatgacaaacaaagaaaaagagttatttgctgaaaatgaggaattaaaaaaagaaattaaggacttaaaagagcgtattgaaagatacagagaaatggaagttgaattaagtacaacaatagatttattgagaggagggattattgaataaataaaagcccccctgacgaaagtcgaagggggcttttattttggtttgatgttgcgattaatagcaatacgattgcaataaacaaaaggatccatgcaagcggcaactgttgtgattaaccgccgcgctctgcgacacaacctgcaacgtcttcgtgaactggcccctgccagtaaaatggttgcggtggtgaaagcgaacgcttatggtcacggtcttcttgagaccgcgcgaacgctccccgatgctgacgcctttggcgtagcccgtctcgaagaagctctgcgactgcgtgcggggggaatcaccaaacctgtactgttactcgaaggcttttttgatgccagagatctgccgacgatttctgcgcaacattttcataccgccgtgcataacgaagaacagctggctgcgctggaagaggctagcctggacgagccggttaccgtctggatgaaactcgataccggtatgcaccgtctgggcgtaaggccggaacaggctgaggcgttttatcatcgcctgacccagtgcaaaaacgttcgtcagccggtgaatatcgtcagccattttgcgcgcgcggatgaaccaaaatgtggcgcaaccgagaaacaactcgctatctttaataccttttgcgaaggcaaacctggtcaacgttccattgccgcgtcgggtggcattctgctgtggccacagtcgcattttgactgggtgcgcccgggcatcattctttatggcgtctcgccgctggaagatcgctccaccggtgccgattttggctgtcagccagtgatgtcactaacctccagcctgattgccgtgcgtgagcataaagccggagagcctgttggttatggtggaacctgggtaagcgaacgtgatacccgtcttggcgtagtcgcgatgggctatggcgatggttatccgcgcgccgcgccgtccggtacgccagtgctggtgaacggtcgcgaagtaccgattgtcgggcgcgtggcgatggatatgatctgcgtagacttaggtccacaggcgcaggacaaagccggggatccggtcattttatggggcgaaggtttgcccgtagaacgtatcgctgaaatgacgaaagtaagcgcttacgaacttattacgcgcctgacttcaagggtcgcgatgaaatacgtggattaaacacgttactaaagggaatggagaccggggcccttcaatagagttcttaacgttaatccgaaaaaaactaacgttaatattaaaaaataagatccgcttgtgaattatgtataatttgattagactaaagaataggagaaagtatgatgatatttaaaaaactttctcgttaagataggttgttggtgagcatgttatatacggatgtatcggtttccttaatgcaaaattttgttgctatcttattaatttttctattatatagatatattcaaagaaagataacatttaaacggatcatattagatattttaatagcgattattttttcaatattatatctgtttatttcagatgcgtcattacttgtaatggtattaatgcgattagggtggcattttcatcaacaaaaagaaaataagataaaaacgactgatacagctaatttaattctaattatcgtgatccagttattgttagttgcggttgggactattattagtcagtttaccatatcgattatcaaaagtgatttcagccaaaatatattgaacaatagtgcaacagatataactttattaggtattttctttgctgttttatttgacggcttgttctttatattattgaagaataagcggactgaattacaacatttaaatcaagaaatcattgaattttcgttagaaaaacaatattttatatttatatttattttatttatagtaatagaaattattttagcagttgggaatcttcaaggagtaacagccacgatattattaaccattatcattattttttgtgtccttatcgggatgactttttggcaagtgatgctttttttgaaggcttattcgattcgccaagaagccaatgaccaattggtccggaatcaacaacttcaagattatctagtcaatatcgaacagcagtacaccgaattacggcgatttaagcatgattatcaaaacatcttattatcgttggagagttttgccgaaaagggcgatcagcaacagtttaaggcgtattaccaagaattattagcacaacggccaattcaaagtgaaatccaaggggcagtcattgcacaactcgactacttgaaaaatgatcctattcgaggattagtcattcaaaagtttttggcagccaaacaggctggtgttactttaaaattcgaaatgaccgaaccaatcgaattagcaaccgctaatctattaacggttattcggattatcggtattttattagacaatgcgattgaacaagccgttcaagaaaccgatcaattggtgagttgtgctttcttacaatctgatggtttaatcgaaattacgattgaaaatacggccagtcaagttaagaatctccaagcattttcagagttaggctattcaacgaaaggcgctggtcgggggactggtttagctaatgtgcaggatttgattgccaaacaaaccaatttattcttagaaacacagattgaaaatagaaagttacgacagacattgatgattacggaggaaacttaatttgtatcccgtttatttattagaggatgatttacagcaacaagcgatttatcagcaaattatcgcgaatacgattatgattaacgaatttgcaatgactttaacatgcgctgccagtgatactgagacattgttggcggcaattaaggatcagcaacgaggtttattctttttggatatggaaattgaggataaccgccaagccggtttagaagtggcaactaagattcggcagatgatgccgtttgcgcaaattgtcttcattacaacccacgaggaactgacattattaacgttagaacgaaaaatagcgcctttagattacattctcaaggaccaaacaatggctgaaatcaaaaggcaattgattgatgatctattgttagctgagaagcaaaacgaggcggcagcgtatcaccgagaaaatttatttagttataaaataggtcctcgctttttctcattaccattaaaggaagttgtttatttatatactgaaaaagaaaatccgggtcatattaatttgttagccgttaccagaaaggttacttttccaggaaatttaaatgcgctggaagcccaatatccaatgctctttcggtgtgataaaagttacttagttaacctatctaatattgccaattatgacagtaaaacacggagtttaaaatttgtagatggcagtgaggcaaaagtctcgttccggaaatcacgggaactagtggccaaattaaaacaaatgatgtagcgcctgcagcacgccaaatgatcccagtaaaaagccacccgcatggcgggtggctttttattagccctagaagggcttcccacacgcatttcagcgccttagtgccttagtttgtgaatcataggtggtatagtcccgaaatacccgtctaaggaattgtcagataggcctaatgactggcttttataatatgagataatgccgactgtactttttacagtcggttttctaatgtcactaacctgccccgttagttgaagaaggtttttatattacagctccagatctaccggtgggcccatattaacgtttaaccgataaagttgaacgttaatattttttttgcgcagaaatggtaaattgaagcataatagtcttgtaaggtatttagctggctggcgtaaagtatgctttataaaataatatataggagtatgattc

SEQ ID NO: 12 - DNA, 39 bp, Artificial
  Terminator iGEM-part BBa_B1006
aaaaaaaaaccccgcccctgacagggcggggtttttttt

SEQ ID NO: 13 - DNA, 7 bp, Artificial
  5′UTR
aggaggt

SEQ ID NO: 14 - DNA, 8 bp, Artificial
  3′UTR
tacttgaa

SEQ ID NO: 15 - DNA, 6603 bp, Artificial
  p3050Alar_Amuc_1100_sh71 with 5′UTR 3′UTR and terminator
atatgaaaaaatttaactttaaaaccatgttgctattagttttggctagttgtgtcttcggggtcgtcgttaacgtgactactagtcttggaccacaaaccgcaatcaccgcccaggcctccaaaggaggtatcgtcaattccaaacgcagtgaactggacaaaaaaatcagcatcgccgccaaggaaatcaagtccgccaatgctgcggaaatcactccgagccgatcatccaacgaagagctggaaaaagaactgaaccgctatgccaaggccgtgggcagcctggaaacggcctacaagcccttccttgcctcctccgcgctggtccccaccacgcccacggcattccagaatgaactgaaaacattcagggattccctgatctcctcctgcaagaaaaagaacattctcataacggacacatcctcctggctcggtttccaggtttacagcacccaggctccctctgttcaggcggcctccacgctgggttttgaattgaaagccatcaacagcctggtcaacaaactggcggaatgcggcctgtccaaattcatcaaggtgtaccgcccccagctccccattgaaaccccggcgaacaatccggaagaatcggacgaagccgaccaggccccatggactcccatgcctctggaaatagccttccagggcgaccgggaaagtgtattgaaagccatgaacgccataaccggcatgcaggactatctgttcacggtcaactccatccgtatccgcaacgaacggatgatgccccctcccatcgccaatccggcagccgccaaacctgccgcggcccaacccgccacgggtgcggcttccctgactccggcggatgaggcggctgcacctgcagccccggccatccagcaagtcatcaagccttacatgggcaaggagcaggtctttgtccaggtctccctgaatctggtccacttcaaccagcccaaggctcaggaaccgtctgaagattaatacttgaaaaaaaaaaaccccgcccctgacagggcggggttttttttcatggatcgattttttattaaaacgtctcaaaatcgtttctgagacgttttagcgtttatttcgtttagttatcggcataatcgttaaaacaggcgttatcgtagcgtaaaagcccttgagcgtagcgtgctttgcagcgaagatgttgtctgttagattatgaaagccgatgactgaatgaaataataagcgcagcgtccttctatttcggttggaggaggctcaagggagtttgagggaatgaaattccctcatgggtttgattttaaaaattgcttgcaattttgccgagcggtagcgctggaaaaatttttgaaaaaaatttggaatttggaaaaaaatggggggaaaggaagcgaattttgcttccgtactacgaccccccattaagtgccgagtgccaatttttgtgccaaaaacgctctatcccaactggctcaagggtttgaggggtttttcaatcgccaacgaatcgccaacgttttcgccaacgttttttataaatctatatttaagtagctttattgttgtttttatgattacaaagtgatacactaattttataaaattatttgattggagttttttaaatggtgatttcagaatcgaaaaaaagagttatgatttctctgacaaaagagcaagataaaaaattaacagatatggcgaaacaaaaaggtttttcaaaatctgcggttgcggcgttagctatagaagaatatgcaagaaaggaatcagaataaaaaaaataagcgaaagctcgcgtttttagaaggatacgagttttcgctacttgtttttgataaggtaatatatcatggctattaaatactaaagctagaaattttggatttttattatatcctgactcaattcctaatgattggaaagaaaaattagagagtttgggcgtatctatggctgtcagtcctttacacgatatggacgaaaaaaaagataaagatacatggaatagtagtgatgttatacgaaatggaaagcactataaaaaaccacactatcacgttatatatattgcacgaaatcctgtaacaatagaaagcgttaggaacaagattaagcgaaaattggggaatagttcagttgctcatgttgagatacttgattatatcaaaggttcatatgaatatttgactcatgaatcaaaggacgctattgctaagaataaacatatatacgacaaaaaagatattttgaacattaatgattttgatattgaccgctatataacacttgatgaaagccaaaaaagagaattgaagaatttacttttagatatagtggatgactataatttggtaaatacaaaagatttaatggcttttattcgccttaggggagcggagtttggaattttaaatacgaatgatgtaaaagatattgtttcaacaaactctagcgcctttagattatggtttgagggcaattatcagtgtggatatagagcaagttatgcaaaggttcttgatgctgaaacgggggaaataaaatgacaaacaaagaaaaagagttatttgctgaaaatgaggaattaaaaaaagaaattaaggacttaaaagagcgtattgaaagatacagagaaatggaagttgaattaagtacaacaatagatttattgagaggagggattattgaataaataaaagcccccctgacgaaagtcgaagggggcttttattttggtttgatgttgcgattaatagcaatacgattgcaataaacaaaaggatccatgcaagcggcaactgttgtgattaaccgccgcgctctgcgacacaacctgcaacgtcttcgtgaactggcccctgccagtaaaatggttgcggtggtgaaagcgaacgcttatggtcacggtcttcttgagaccgcgcgaacgctccccgatgctgacgcctttggcgtagcccgtctcgaagaagctctgcgactgcgtgcggggggaatcaccaaacctgtactgttactcgaaggcttttttgatgccagagatctgccgacgatttctgcgcaacattttcataccgccgtgcataacgaagaacagctggctgcgctggaagaggctagcctggacgagccggttaccgtctggatgaaactcgataccggtatgcaccgtctgggcgtaaggccggaacaggctgaggcgttttatcatcgcctgacccagtgcaaaaacgttcgtcagccggtgaatatcgtcagccattttgcgcgcgcggatgaaccaaaatgtggcgcaaccgagaaacaactcgctatctttaataccttttgcgaaggcaaacctggtcaacgttccattgccgcgtcgggtggcattctgctgtggccacagtcgcattttgactgggtgcgcccgggcatcattctttatggcgtctcgccgctggaagatcgctccaccggtgccgattttggctgtcagccagtgatgtcactaacctccagcctgattgccgtgcgtgagcataaagccggagagcctgttggttatggtggaacctgggtaagcgaacgtgatacccgtcttggcgtagtcgcgatgggctatggcgatggttatccgcgcgccgcgccgtccggtacgccagtgctggtgaacggtcgcgaagtaccgattgtcgggcgcgtggcgatggatatgatctgcgtagacttaggtccacaggcgcaggacaaagccggggatccggtcattttatggggcgaaggtttgcccgtagaacgtatcgctgaaatgacgaaagtaagcgcttacgaacttattacgcgcctgacttcaagggtcgcgatgaaatacgtggattaaacacgttactaaagggaatggagaccggggcccttcaatagagttcttaacgttaatccgaaaaaaactaacgttaatattaaaaaataagatccgcttgtgaattatgtataatttgattagactaaagaataggagaaagtatgatgatatttaaaaaactttctcgttaagataggttgttggtgagcatgttatatacggatgtatcggtttccttaatgcaaaattttgttgctatcttattaatttttctattatatagatatattcaaagaaagataacatttaaacggatcatattagatattttaatagcgattattttttcaatattatatctgtttatttcagatgcgtcattacttgtaatggtattaatgcgattagggtggcattttcatcaacaaaaagaaaataagataaaaacgactgatacagctaatttaattctaattatcgtgatccagttattgttagttgcggttgggactattattagtcagtttaccatatcgattatcaaaagtgatttcagccaaaatatattgaacaatagtgcaacagatataactttattaggtattttctttgctgttttatttgacggcttgttctttatattattgaagaataagcggactgaattacaacatttaaatcaagaaatcattgaattttcgttagaaaaacaatattttatatttatatttattttatttatagtaatagaaattattttagcagttgggaatcttcaaggagtaacagccacgatattattaaccattatcattattttttgtgtccttatcgggatgactttttggcaagtgatgctttttttgaaggcttattcgattcgccaagaagccaatgaccaattggtccggaatcaacaacttcaagattatctagtcaatatcgaacagcagtacaccgaattacggcgatttaagcatgattatcaaaacatcttattatcgttggagagttttgccgaaaagggcgatcagcaacagtttaaggcgtattaccaagaattattagcacaacggccaattcaaagtgaaatccaaggggcagtcattgcacaactcgactacttgaaaaatgatcctattcgaggattagtcattcaaaagtttttggcagccaaacaggctggtgttactttaaaattcgaaatgaccgaaccaatcgaattagcaaccgctaatctattaacggttattcggattatcggtattttattagacaatgcgattgaacaagccgttcaagaaaccgatcaattggtgagttgtgctttcttacaatctgatggtttaatcgaaattacgattgaaaatacggccagtcaagttaagaatctccaagcattttcagagttaggctattcaacgaaaggcgctggtcgggggactggtttagctaatgtgcaggatttgattgccaaacaaaccaatttattcttagaaacacagattgaaaatagaaagttacgacagacattgatgattacggaggaaacttaatttgtatcccgtttatttattagaggatgatttacagcaacaagcgatttatcagcaaattatcgcgaatacgattatgattaacgaatttgcaatgactttaacatgcgctgccagtgatactgagacattgttggcggcaattaaggatcagcaacgaggtttattctttttggatatggaaattgaggataaccgccaagccggtttagaagtggcaactaagattcggcagatgatgccgtttgcgcaaattgtcttcattacaacccacgaggaactgacattattaacgttagaacgaaaaatagcgcctttagattacattctcaaggaccaaacaatggctgaaatcaaaaggcaattgattgatgatctattgttagctgagaagcaaaacgaggcggcagcgtatcaccgagaaaatttatttagttataaaataggtcctcgctttttctcattaccattaaaggaagttgtttatttatatactgaaaaagaaaatccgggtcatattaatttgttagccgttaccagaaaggttacttttccaggaaatttaaatgcgctggaagcccaatatccaatgctctttcggtgtgataaaagttacttagttaacctatctaatattgccaattatgacagtaaaacacggagtttaaaatttgtagatggcagtgaggcaaaagtctcgttccggaaatcacgggaactagtggccaaattaaaacaaatgatgtagcgcctgcagcacgccaaatgatcccagtaaaaagccacccgcatggcgggtggctttttattagccctagaagggcttcccacacgcatttcagcgccttagtgccttagtttgtgaatcataggtggtatagtcccgaaatacccgtctaaggaattgtcagataggcctaatgactggcttttataatatgagataatgccgactgtactttttacagtcggttttctaatgtcactaacctgccccgttagttgaagaaggtttttatattacagctccagatctaccggtgggcccatattaacgtttaaccgataaagttgaacgttaatattttttttgcgcagaaatggtaaattgaagcataatagtcttgtaaggtatttagctggctggcgtaaagtatgctttataaaataatatataggagtatgattc

(19) United States
(12) Patent Application Publication
(10) Pub. No.: US 2021/0322492 A1
(43) Pub. Date: Oct. 21, 2021

(54) PROBIOTIC STRAINS AND USES THEREOF

(71) Applicant: AMUCIN OY LTD, Helsinki (FI)

(72) Inventor: Ville TAKIO, Helsinki (FI)

(21) Appl. No.: 16/850,415

(22) Filed: Apr. 16, 2020

(51) Int. Cl. A61K 35/747; A23L 33/135; A61K 35/745; A61K 35/742; C12N 15/74; A23C 9/123

(52) U.S. Cl. CPC A61K 35/747; A23L 33/135; A61K 35/745; A61K 9/0053; C12N 15/746; A23C 9/1234; C12Y 501/01001; A61K 35/742

(57) ABSTRACT

The present invention pertains to a probiotic comprising a generally recognized as safe (GRAS) microbiological organism, which GRAS microbiological organism comprises a food-grade expression vector, which vector comprises in functional linkage a nucleic acid sequence encoding for a soluble form of Amuc_1100 or a functionally equivalent fragment of said soluble form of Amuc_1100, wherein said GRAS microbiological organism is capable of expressing and secreting said soluble form of Amuc_1100 or said fragment thereof, as further defined in the claims. Methods for treating a disease in a patient, comprising oral administration of the probiotic as defined herein are also described, as well as methods of preparing the probiotic disclosed herein.

The present invention pertains to a probiotic comprising a generally recognized as safe (GRAS) microbiological organism, which GRAS microbiological organism comprises a food-grade expression vector, which vector comprises in functional linkage a nucleic acid sequence encoding for a soluble form of Amuc_1100 or a functionally equivalent fragment of said soluble form of Amuc_1100, wherein said GRAS microbiological organism is capable of expressing and secreting said soluble form of Amuc_1100 or said fragment thereof, as further defined in the claims. Methods for treating a disease in a patient, comprising oral administration of the probiotic as defined herein are also described, as well as methods of preparing the probiotic disclosed herein.

### REFERENCE TO AN ELECTRONIC SEQUENCE LISTING

The contents of the electronic sequence listing (227-361_Sequence_Listing.txt; Size: 49,937 bytes; and Date of Creation: Apr. 15, 2020) is herein incorporated by reference in its entirety.

### BACKGROUND OF THE INVENTION

In 2001, the World Health organization (WHO) defined in a report probiotics as live microorganisms that, “when administered in adequate amounts, confer a health benefit on the host.” Following this definition, a working group of the Food and Agriculture Organization (FAO)/WHO issued the Guidelines for the Evaluation of Probiotics in Food in 2002. A consensus definition of the term probiotics, based on available information and scientific evidence, was adopted after the aforementioned joint expert consultation between the FAO of the United Nations and the WHO.

The National Center for Complementary and Integrative Health describe probiotics as live microorganisms that are intended to have health benefits when consumed or applied to the body. They are usually provided in form of yoghurt and other fermented foods, dietary supplements, and beauty products. Some bacteria are considered to help in digesting food, destroy disease-causing cells, or produce vitamins. Administration of probiotics is intended to induce changes in the microbiome in the gut, often in order to promote growth of microorganisms which are considered beneficial over those which are considered detrimental. Another mode of action of probiotics is considered by interactions between the probiotic microorganism and the host.

Ottman et al. (PLOS ONE (2017), 12(3): e0173004; doi:10.1371/journal.phone.0173004) disclose that the gut symbiont Akkermansia muciniphilia is positively correlated with a lean physiology, reduced body weight gain, amelioration or metabolic responses and restoration of gut barrier function by modulation of mucus layer thickness. The authors identified some of these beneficial effects to be due to an outer membrane pili-like protein named Amuc_1100. When expressed in a non food-grade expression vector as a purification-tagged protein in the non-GRAS microorganism E. coli, and following its purification, the purified protein was found to be a strong TLR2 activator and inducer of inter alia IL-10. Ottman et al. finally suggest the use of gram-negative Akkermansia muciniphilia as a probiotic.

Similarly, Plovier et al. (Nature Medicine 2016; doi: 10.10387 nm.4236) report that a purified His-tagged form of the membrane protein Amuc_1100 from Akkermansia muciniphila (expressed in E. coli) or the pasteurized Akkermansia muciniphila bacterium improves metabolism in obese and diabetic mice. Plovier et al. conclude that either live or pasteurized A. muciniphila (i.e. the bacterium) grown on synthetic medium are a promising therapeutic tool in the management of metabolic syndrome.

Toll-like receptor 2 (TLR2), also designated as CD282, is a receptor of the Toll-like receptor (TLR) family, which plays a fundamental role in the recognition of pathogen-associated molecular patterns (PAMPs) that are expressed on infectious agents. Upon activation, TLRs mediate the production of cytokines necessary for modulating the immune response. TLR2 is expressed most abundantly in peripheral blood leukocytes, and mediates host response to mainly gram-positive bacteria, and yeast via stimulation of NF-κB. However, TLR2 recognizes many bacterial, viral and fungal compounds, as well as certain endogenous substances. In the intestine, TLR2 regulates the expression of CYP1A1, an enzyme which is key in detoxication of certain carcinogenic substances. Recently, it was found that TLR2 is involved in the activation of regulatory T cells (Tregs), that act to suppress immune response, thereby maintaining homeostasis and self-tolerance. It has been shown that Tregs are able to inhibit T cell proliferation and cytokine production and play a critical role in preventing autoimmunity. TLR2 is also expressed by intestinal epithelial cells and subsets of lamina propria mononuclear cells in the gastrointestinal tract. TLR2 has been observed downregulated in human papillomavirus-positive neoplastic keratocytres derived from uterine cervical preneoplastic lesions. Thus, TLR2 is assumed to be associated with tumorigenesis.

Often the microorganisms in probiotic foods are the same or similar to the ones naturally abundant in the human body. In contrast thereto, prebiotics are non-digestable food components that selectively stimulate the growth or activity of certain microorganisms. The term synbiotics commonly refers to products that combine probiotics and prebiotics.

Nguyen et al. (J. Agric. Food Chem. 2011, 59, 5617-5624) discloses a food-grade system for inducible gene expression in Lactobacillus plantarum.

In 2015, the global retail market value for probiotics was US$41 billion, including sales of probiotic dietary supplements, fermented dairy products, and yoghurt, the latter accounting for 75% of total consumption. In 2015 supplements produced US$4 billion and their growth is projected to be as high as 37% globally by 2020. At the same time, consumption of probiotic yoghurt in China has increased by 20% per year since 2014.

There is an existing need in the art for new useful probiotics, which exhibit and combine beneficial health effects. Such probiotics may suitably be applied in the treatment of diseases, including obesity and diabetes.

### BRIEF EXPLANATION OF THE INVENTION

The aforementioned need is addressed by the present invention, which is characterized by improving the health benefit of a generally recognized as safe (GRAS) microbiological organism, by incorporating into said GRAS microbiological organism a food-grade expression vector, which vector comprises in functional linkage a nucleic acid sequence encoding for a soluble form of Amuc_1100 or a functionally equivalent fragment of said soluble form of Amuc_1100, such that the GRAS microbiological organism is capable of expressing and secreting said soluble form of Amuc_1100 or said fragment thereof.

The invention is particularly advantageous for embodiments, wherein the GRAS microbiological organism is selected from the group of organisms consisting of a gram-positive bacteria and a gram-negative bacteria. This is because it is expected that the beneficial effects reported for Amuc_1100, in particular its Toll-like receptor 2 (TLR-2) agonistic activity, will further improve the beneficial health effects which are ascribed to the induction of TLR-2 by PAMPs found in the membrane of these microorganisms. A particular advantageous benefit is to be expected in embodiments, wherein the GRAS microbiological organism is a gram-positive bacteria belonging to the order of lactic acid bacteria.

To the Applicant's best knowledge, there is no suggestion in the prior art to express a soluble form of Amuc_1100, or a functionally equivalent soluble fragment thereof, in a probiotic GRAS microbiological organism, let alone in a GRAS microorganism of the embodiments described herein. Rather, prior to the present invention, it was suggested to use live or pasteurized Akkermansia muciniphila. However, in the context of the invention, the GRAS microorganism is not Akkermansia muciniphila. In the alternative, a His-tagged Amuc_1100 protein was produced in E. coli and used in purified form for research purposes. In contrast, in embodiments of the present invention, said soluble form of Amuc_1100 or a fragment of said soluble form of Amuc_1100 does not need to comprise such a purification tag, and need not to be purified.

Moreover, while food-grade expression systems are disclosed for primary use in organisms of the genus Lactobacillus, in embodiments these expression systems are used in genus other than Lactobacillus, where these food-grade expression vectors are also functional. In this context, in embodiments said food-grade expression vector carries the SH71rep replicon, which has a broad functionality. Usually, said food-grade expression vector may carry a food-grade selection marker, which provides prototrophy to an otherwise auxotroph GRAS microbiological organism. In embodiments, the marker is alanine racemase (alr).

In embodiments, the nucleic acid sequence in said food-grade expression vector encodes a soluble form of Amuc_1100 having an amino acid sequence with at least 80% identity to SEQ ID NO: 2 (Amuc_1100). In embodiments, said nucleic acid sequence encodes for a fragment of said soluble form of Amuc_1100, which has a length of at least 100 and up to 286 amino acids. Said nucleic acid sequence may also be optimized for expression in the genus selected from the group of Bifidobacterium, Bacillus, Brevibacillus, Lactococcus and Saccharomyces. Hence, in embodiments said nucleic acid sequence has at least 70% identity to SEQ ID NO: 1 (Amuc_1100). One useful example of said food-grade expression vector is p3050alrAmuc_1100-sh71 (SEQ ID NO: 9) or p3050Alr_Amuc1100-sh71 with 5′UTR, 3′UTR and terminator (SEQ ID NO: 15).

In a further aspect, the present invention also pertains to a method of treating a disease in a patient, comprising the step of administering orally a probiotic of the present invention. In embodiments, the disease is selected from the group consisting of obesity, diabetes, hypercholesterolemia, and/or the patient is a human patient.

Further provided is a method for preparing a prebiotic according to the present invention, wherein the method comprises the step of introducing a food-grade expression vector, which vector comprises in functional linkage a nucleic acid sequence encoding for a soluble form of Amuc_1100 or a functionally equivalent fragment of said soluble form of Amuc_1100, into a GRAS microbiological organism, such that said GRAS microbiological organism is capable of expressing and secreting said soluble form of Amuc_1100 or said fragment thereof.

Other objectives, aspects, embodiments, details and advantages of the present invention will become apparent from the following figures, detailed description, examples, and dependent claims.

### BRIEF DESCRIPTION OF THE DRAWINGS

FIG. 1 shows a vector map of p3050alarAmuc1100-sh71 (SEQ ID NO: 9).

FIG. 2 shows a vector map of p3050alarAmuc1100_alcA-al1b1-sh71 (SEQ ID NO: 11).

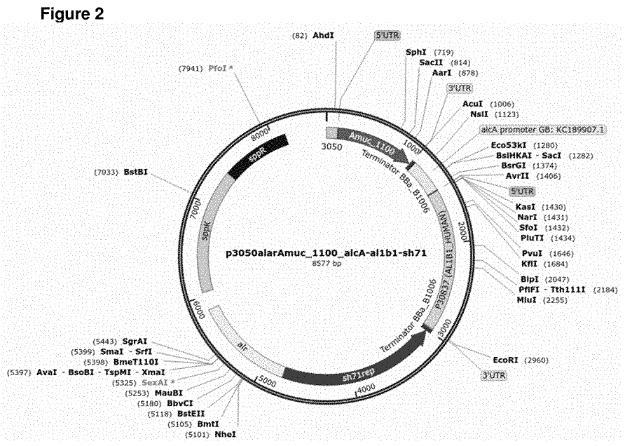

FIG. 3 shows a vector map of p3050Alr_Amuc1100_sh71 with 5′UTR, 3′UTR and terminator (SEQ ID NO: 15).

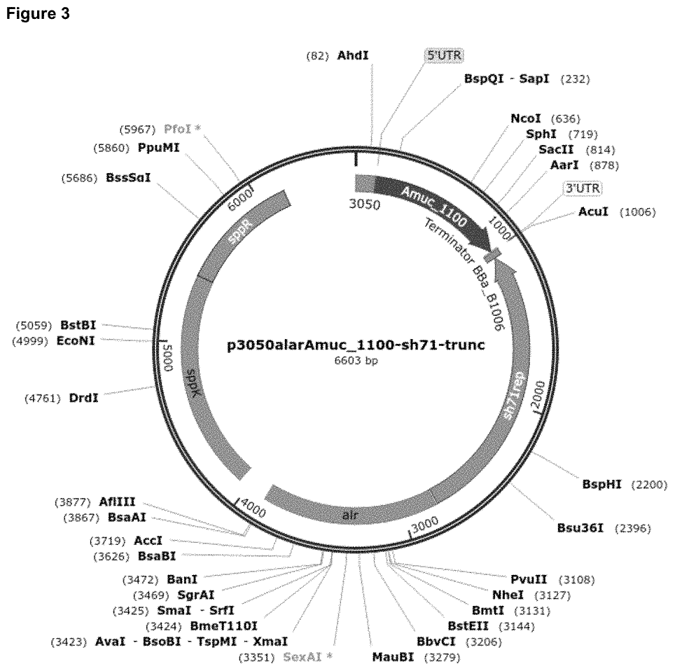

### BRIEF DESCRIPTION OF THE SEQUENCES

| Nucleic acid sequence encoding Amuc_1100 without its signal sequence | |
|---|---|
| (aa 1-30), (SEQ ID NO: 1): | |
| atcgtcaatt ccaaacgcag tgaactggac aaaaaaatca gcatcgccgc caaggaaatc | 60 |
| aagtccgcca atgctgcgga aatcactccg agccgatcat ccaacgaaga gctggaaaaa | 120 |
| gaactgaacc gctatgccaa ggccgtgggc agcctggaaa cggcctacaa gcccttcctt | 180 |
| gcctcctccg cgctggtccc caccacgccc acggcattcc agaatgaact gaaaacattc | 240 |
| agggattccc tgatctcctc ctgcaagaaa aagaacattc tcataacgga cacatcctcc | 300 |
| tggctcggtt tccaggttta cagcacccag gctccctctg ttcaggcggc ctccacgctg | 360 |
| ggttttgaat tgaaagccat caacagcctg gtcaacaaac tggcggaatg cggcctgtcc | 420 |
| aaattcatca aggtgtaccg cccccagctc cccattgaaa ccccggcgaa caatccggaa | 480 |
| gaatcggacg aagccgacca ggccccatgg actcccatgc ctctggaaat agccttccag | 540 |
| ggcgaccggg aaagtgtatt gaaagccatg aacgccataa ccggcatgca ggactatctg | 600 |
| ttcacggtca actccatccg tatccgcaac gaacggatga tgccccctcc catcgccaat | 660 |
| ccggcagccg ccaaacctgc cgcggcccaa cccgccacgg gtgcggcttc cctgactccg | 720 |
| gcggatgagg cggctgcacc tgcagccccg gccatccagc aagtcatcaa gccttacatg | 780 |
| ggcaaggagc aggtctttgt ccaggtctcc ctgaatctgg tccacttcaa ccagcccaag | 840 |
| gctcaggaac cgtctgaaga ttaa | 864 |
| Amino acid sequence of Amuc_1100 without its signal sequence (aa 1-30), | |
| (SEQ ID NO: 2): | |
| I V N S K R S E L D K K I S I A A K E I K S A N A | |
| A E I T P S R S S N E E L E K E L N R Y A K A V G | |
| S L E T A Y K P F L A S S A L V P T T P T A F Q N | |
| E L K T F R D S L I S S C K K K N I L I T D T S S | |
| W L G F Q V Y S T Q A P S V Q A A S T L G F E L K | |
| A I N S L V N K L A E C G L S K F I K V Y R P Q L | |
| P I E T P A N N P E E S D E A D Q A P W T P M P L | |
| E I A F Q G D R E S V L K A M N A I T G M Q D Y L | |
| F T V N S I R I R N E R M M P P P I A N P A A A K | |
| P A A A Q P A T G A A S L T P A D E A A A P A A P | |
| A I Q Q V I K P Y M G K E Q V F V Q V S L N L V H | |
| F N Q P K A Q E P S E D | |
| Amuc_1100 Δ1-30 sequence optimized for Bifidobacterium (SEQ ID NO: 3): | |
| attgtgaact ccaagcgctc cgagctggac aagaagatca gcattgccgc taaggagatc | 60 |
| aagtccgcca atgctgccga gatcacgccc tccaggagca gcaacgagga gctggaaaag | 120 |
| gagctgaacc ggtatgccaa agcggtgggt agcctggaaa ccgcgtacaa acccttcctt | 180 |
| gcgtcctcgg cgctcgttcc gaccaccccg acggccttcc agaacgagct caagacgttc | 240 |
| cgcgactccc tcatctcgtc ctgcaagaag aagaacatcc tcatcaccga tacgagctcc | 300 |
| tggttgggct tccaggtgta ctccacccag gccccgtcgg tccaagccgc ctcgaccttg | 360 |
| ggcttcgaac tgaaggccat caactccctg gtgaacaagc tggccgaatg cgggctgtcc | 420 |
| aagttcatca aggtgtatcg tccgcagctc cccatcgaaa ccccggccaa caaccccgag | 480 |
| gaatccgacg aggccgatca ggcgccctgg accccgatgc ctctcgagat cgcctttcag | 540 |
| ggcgatcgcg agtccgtgct gaaggcgatg aacgccatca ccggcatgca ggactacctt | 600 |
| ttcacggtga acagcatccg catccggaac gagcgcatga tgccgccgcc gattgcgaat | 660 |
| ccggcggccg cgaaaccggc agctgcccaa ccggccactg gagcagccag cctgacccct | 720 |
| gcggacgagg cagccgctcc tgcagctccg gcgatccaac aggtcatcaa gccgtacatg | 780 |
| ggcaaggaac aggtgttcgt ccaggtttcc ctgaacctgg tccacttcaa ccagcccaaa | 840 |
| gcccaggaac cgtcggagga ctga | 864 |
| Amuc_1100 Δ1-30 sequence optimized for Bacillus species (SEQ ID NO: 4): | |
| attgtgaact caaaacggtc tgagttggac aagaaaatca gcatagctgc aaaagagatc | 60 |
| aaatccgcaa acgcagcaga aattacgccg tcaagaagtt ccaacgaaga gctggagaaa | 120 |
| gaactgaatc gctatgccaa agcggttgga tcacttgaaa cggcatacaa gccgtttctt | 180 |
| gcgagctctg cccttgtacc gacaacaccg acagcgttcc aaaacgaact gaaaacattt | 240 |
| cgtgacagcc ttatatcttc ctgcaagaag aagaacatcc tcatcactga tacaagctct | 300 |
| tggttaggct ttcaggtgta tagcacacaa gcaccttcag ttcaagcggc atcaacgtta | 360 |
| ggctttgagc tgaaagccat caattcgttg gtgaacaaac ttgcggaatg tggcttatcg | 420 |
| aagtttatca aagtctatcg tccgcaatta cccattgaaa ccccagcaaa taaccctgaa | 480 |
| gaatcggatg aggcggatca agccccttgg accccaatgc ctttggaaat tgcctttcag | 540 |
| ggtgatagag aatctgtttt aaaagccatg aatgcgatta ccggaatgca ggactatctg | 600 |
| ttcacggtca atagtattcg cattcgaaat gagaggatga tgccaccgcc gattgctaat | 660 |
| cctgcagccg ctaaaccagc tgctgctcaa ccggcaactg gagctgcaag tctgactcct | 720 |
| gcggatgaag cggctgctcc agctgcccct gcgattcaac aggtaatcaa accgtacatg | 780 |
| gggaaagaac aggtatttgt ccaggtttca ttgaatctcg tgcatttcaa tcagccgaaa | 840 |
| gcccaagaac ccagcgaaga ttaa | 864 |
| Amuc_1100 AΔ-30 sequence optimized for Brevibacillus species (SEQ ID NO: | |
| 5): | |
| atcgtcaata gcaaacgcag tgaactggac aagaaaatct ccattgccgc aaaagagatc | 60 |
| aaatccgcaa acgctgccga aatcactccc tctcgtagtt ctaacgagga actggagaaa | 120 |
| gaactgaatc gctatgctaa agccgtaggc tctctggaaa ccgcgtacaa accgtttctt | 180 |
| gcgtcctctg cattggtccc caccacaccg accgcgtttc agaatgagct gaaaaccttc | 240 |
| cgcgattctc tgatctcgag ctgcaagaag aagaacatcc tcatcaccga cacatcgtcc | 300 |
| tggttgggat tccaagtata ctccacgcaa gctccaagcg tacaagcggc atcgactctt | 360 |
| ggctttgagc tgaaagctat caactccctc gttaacaagc tcgcggagtg tggcctttcc | 420 |
| aaattcatca aggtgtatcg acctcagctg ccaatcgaaa ctccggctaa caaccctgaa | 480 |
| gaatccgatg aagcagatca agccccatgg actccgatgc cactggaaat cgcgtttcaa | 540 |
| ggtgaccgtg aatccgtact gaaagccatg aacgcaatca cggggatgca agactacttg | 600 |
| ttcacggtga actccattcg cattcgcaat gaacgcatga tgccacctcc aattgcgaat | 660 |
| cctgcagctg caaaaccagc tgcggcacaa cccgctacag gtgcggcatc cttgactccg | 720 |
| gcagacgaag ctgctgctcc agctgcgcct gcaatccagc aagtgatcaa accctatatg | 780 |
| ggcaaagaac aggttttcgt acaggtttcc ctgaatctgg tgcatttcaa ccaaccgaaa | 840 |
| gcgcaagaac cttccgaaga ttaa | 864 |
| Amuc_1100 Δ1-30 sequence optimized for Lactococcus species (SEQ ID NO: | |
| 6): | |
| atagttaaca gcaaacgatc agagttagac aagaaaattt caattgcagc aaaggagata | 60 |
| aaatctgcca atgctgctga gattactccc tctagaagtt caaacgaaga acttgagaaa | 120 |
| gaattgaata gatatgcgaa agcggttggt tcacttgaaa ccgcgtataa accgtttcta | 180 |
| gcgagttctg ccttagtacc aactacacca acggcatttc agaatgaact taaaactttt | 240 |
| agagacagct taatttcatc atgcaagaag aagaacatac ttattacaga tacctcatca | 300 |
| tggttaggat ttcaggttta tagtactcaa gctccttcag ttcaagccgc atcaacgttg | 360 |
| ggttttgagt tgaaagcgat taatagctta gtaaacaaac ttgctgaatg tgggttgagt | 420 |
| aaatttatca aagtctatag accgcaatta cctattgaaa ctcccgctaa taatccagaa | 480 |
| gaaagtgatg aagcagatca agcaccatgg acacctatgc ctttggaaat tgcctttcaa | 540 |
| ggagatcgag aaagtgtttt aaaagccatg aatgcaatta caggaatgca agattactta | 600 |
| ttcaccgtca attctattcg tatccgtaat gaacgcatga tgcctccacc tattgcaaat | 660 |
| cctgcagctg ctaaaccggc tgcagcacaa ccagctacag gtgcagcttc tctaacacca | 720 |
| gccgatgaag ctgctgctcc agctgcacca gccatacaac aggtaatcaa accttatatg | 780 |
| ggcaaagaac aagtgtttgt tcaagtgtct ttaaatttag ttcatttcaa tcaaccaaaa | 840 |
| gctcaagaac catcagaaga ttaa | 864 |
| Amuc_1100 Δ1-30 sequence optimized for Saccharomyces (SEQ ID NO: 7): | |
| attgttaatt ctaagagatc cgaactggac aagaaaatctcgattgcagc gaaggaaatc | 60 |
| aaatcggcta atgcagctga aatcactcct tcaaggtctagtaacgagga attggagaaa | 120 |
| gaattgaaca gatatgctaa agcagttggt agcttggaaacagcctataa accgttctta | 180 |
| gcatctagcg cattagttcc aaccactcca acagcgtttcagaatgaact gaaaacgttt | 240 |
| agagacagct tgattagttc ttgcaagaag aagaacatcttgataacaga caccagttca | 300 |
| tggttaggct ttcaagtata ctctactcaa gcaccatcagttcaagctgc atccactttg | 360 |
| ggattcgagt taaaggccat aaactcactt gtgaacaaacttgctgaatg tggtctatcc | 420 |
| aagttcatca aagtttacag accccagtta ccgattgaaactcccgcaaa taatcctgaa | 480 |
| gagtcagatg aagccgatca agctccttgg acacctatgcctctagaaat tgcttttcag | 540 |
| ggtgatagag agagtgtatt gaaagcgatg aatgccattacaggtatgca agattaccta | 600 |
| tttaccgtaa attccattag gatacgtaac gagagaatgatgccaccacc aattgccaat | 660 |
| cctgctgcag ccaaacccgc tgccgctcaa ccagcgactggagcagcatc tcttacgcca | 720 |
| gccgatgaag ctgcagctcc agctgctcct gccatacaacaggtgataaa accctatatg | 780 |
| gggaaagaac aggtctttgt ccaagtctcg ttgaatttagtgcatttcaa ccaaccaaag | 840 |
| gctcaagaac cgtctgagga ttaa | 864 |
| Alanine racemase (alr) | |
| (SEQ ID NO: 8) | |
| atgcaagcgg caactgttgt gattaaccgc cgcgctctgc gacacaacct gcaacgtctt | 60 |
| cgtgaactgg cccctgccag taaaatggtt gcggtggtga aagcgaacgc ttatggtcac | 120 |
| ggtcttcttg agaccgcgcg aacgctcccc gatgctgacg cctttggcgt agcccgtctc | 180 |
| gaagaagctc tgcgactgcg tgcgggggga atcaccaaac ctgtactgtt actcgaaggc | 240 |
| ttttttgatg ccagagatct gccgacgatt tctgcgcaac attttcatac cgccgtgcat | 300 |
| aacgaagaac agctggctgc gctggaagag gctagcctgg acgagccggt taccgtctgg | 360 |
| atgaaactcg ataccggtat gcaccgtctg ggcgtaaggc cggaacaggc tgaggcgttt | 420 |
| tatcatcgcc tgacccagtg caaaaacgtt cgtcagccgg tgaatatcgt cagccatttt | 480 |
| gcgcgcgcgg atgaaccaaa atgtggcgca accgagaaac aactcgctat ctttaatacc | 540 |
| ttttgcgaag gcaaacctgg tcaacgttcc attgccgcgt cgggtggcat tctgctgtgg | 600 |
| ccacagtcgc attttgactg ggtgcgcccg ggcatcattc tttatggcgt ctcgccgctg | 660 |
| gaagatcgct ccaccggtgc cgattttggc tgtcagccag tgatgtcact aacctccagc | 720 |
| ctgattgccg tgcgtgagca taaagccgga gagcctgttg gttatggtgg aacctgggta | 780 |
| agcgaacgtg atacccgtct tggcgtagtc gcgatgggct atggcgatgg ttatccgcgc | 840 |
| gccgcgccgt ccggtacgcc agtgctggtg aacggtcgcg aagtaccgat tgtcgggcgc | 900 |
| gtggcgatgg atatgatctg cgtagactta ggtccacagg cgcaggacaa agccggggat | 960 |
| ccggtcattt tatggggcga aggtttgccc gtagaacgta tcgctgaaat gacgaaagta | 1020 |
| agcgcttacg aacttattac gcgcctgact tcaagggtcg cgatgaaata cgtggattaa | 1080 |
| p3050Alr_Amuc1100_sh71 | |
| (SEQ ID NO: 9) | |
| atatgaaaaa atttaacttt aaaaccatgt tgctattagt tttggctagt tgtgtcttcg | 60 |
| gggtcgtcgt taacgtgact actagtcttg gaccacaaac cgcaatcacc gcccaggcct | 120 |
| ccaaggtcga catcgtcaat tccaaacgca gtgaactgga caaaaaaatc agcatcgccg | 180 |
| ccaaggaaat caagtccgcc aatgctgcgg aaatcactcc gagccgatca tccaacgaag | 240 |
| agctggaaaa agaactgaac cgctatgcca aggccgtggg cagcctggaa acggcctaca | 300 |
| agcccttcct tgcctcctcc gcgctggtcc ccaccacgcc cacggcattc cagaatgaac | 360 |
| tgaaaacatt cagggattcc ctgatctcct cctgcaagaa aaagaacatt ctcataacgg | 420 |
| acacatcctc ctggctcggt ttccaggttt acagcaccca ggctccctct gttcaggcgg | 480 |
| cctccacgct gggttttgaa ttgaaagcca tcaacagcct ggtcaacaaa ctggcggaat | 540 |
| gcggcctgtc caaattcatc aaggtgtacc gcccccagct ccccattgaa accccggcga | 600 |
| acaatccgga agaatcggac gaagccgacc aggccccatg gactcccatg cctctggaaa | 660 |
| tagccttcca gggcgaccgg gaaagtgtat tgaaagccat gaacgccata accggcatgc | 720 |
| aggactatct gttcacggtc aactccatcc gtatccgcaa cgaacggatg atgccccctc | 780 |
| ccatcgccaa tccggcagcc gccaaacctg ccgcggccca acccgccacg ggtgcggctt | 840 |
| ccctgactcc ggcggatgag gcggctgcac ctgcagcccc ggccatccag caagtcatca | 900 |
| agccttacat gggcaaggag caggtctttg tccaggtctc cctgaatctg gtccacttca | 960 |
| accagcccaa ggctcaggaa ccgtctgaag attaaaagct tcaaattaca gcacgtgttg | 1020 |
| ctttgattga tagccaaaaa gcagcagttg ataaagcaat tactgatatt gctgaaaaat | 1080 |
| tgtaatttat aaataaaaat caccttttag aggtggtttt tttatttata aattattcgt | 1140 |
| ttgatttcgc tttcgataga acaatcaaag cgagaataag gaagataaat cccataaggg | 1200 |
| cgggagcaga atgtccgaga ctaattcatg gatcgatttt ttattaaaac gtctcaaaat | 1260 |
| cgtttctgag acgttttagc gtttatttcg tttagttatc ggcataatcg ttaaaacagg | 1320 |
| cgttatcgta gcgtaaaagc ccttgagcgt agcgtgcttt gcagcgaaga tgttgtctgt | 1380 |
| tagattatga aagccgatga ctgaatgaaa taataagcgc agcgtccttc tatttcggtt | 1440 |
| ggaggaggct caagggagtt tgagggaatg aaattccctc atgggtttga ttttaaaaat | 1500 |
| tgcttgcaat tttgccgagc ggtagcgctg gaaaaatttt tgaaaaaaat ttggaatttg | 1560 |
| gaaaaaaatg gggggaaagg aagcgaattt tgcttccgta ctacgacccc ccattaagtg | 1620 |
| ccgagtgcca atttttgtgc caaaaacgct ctatcccaac tggctcaagg gtttgagggg | 1680 |
| tttttcaatc gccaacgaat cgccaacgtt ttcgccaacg ttttttataa atctatattt | 1740 |
| aagtagcttt attgttgttt ttatgattac aaagtgatac actaatttta taaaattatt | 1800 |
| tgattggagt tttttaaatg gtgatttcag aatcgaaaaa aagagttatg atttctctga | 1860 |
| caaaagagca agataaaaaa ttaacagata tggcgaaaca aaaaggtttt tcaaaatctg | 1920 |
| cggttgcggc gttagctata gaagaatatg caagaaagga atcagaataa aaaaaataag | 1980 |
| cgaaagctcg cgtttttaga aggatacgag ttttcgctac ttgtttttga taaggtaata | 2040 |
| tatcatggct attaaatact aaagctagaa attttggatt tttattatat cctgactcaa | 2100 |
| ttcctaatga ttggaaagaa aaattagaga gtttgggcgt atctatggct gtcagtcctt | 2160 |
| tacacgatat ggacgaaaaa aaagataaag atacatggaa tagtagtgat gttatacgaa | 2220 |
| atggaaagca ctataaaaaa ccacactatc acgttatata tattgcacga aatcctgtaa | 2280 |
| caatagaaag cgttaggaac aagattaagc gaaaattggg gaatagttca gttgctcatg | 2340 |
| ttgagatact tgattatatc aaaggttcat atgaatattt gactcatgaa tcaaaggacg | 2400 |
| ctattgctaa gaataaacat atatacgaca aaaaagatat tttgaacatt aatgattttg | 2460 |
| atattgaccg ctatataaca cttgatgaaa gccaaaaaag agaattgaag aatttacttt | 2520 |
| tagatatagt ggatgactat aatttggtaa atacaaaaga tttaatggct tttattcgcc | 2580 |
| ttaggggagc ggagtttgga attttaaata cgaatgatgt aaaagatatt gtttcaacaa | 2640 |
| actctagcgc ctttagatta tggtttgagg gcaattatca gtgtggatat agagcaagtt | 2700 |
| atgcaaaggt tcttgatgct gaaacggggg aaataaaatg acaaacaaag aaaaagagtt | 2760 |
| atttgctgaa aatgaggaat taaaaaaaga aattaaggac ttaaaagagc gtattgaaag | 2820 |
| atacagagaa atggaagttg aattaagtac aacaatagat ttattgagag gagggattat | 2880 |
| tgaataaata aaagcccccc tgacgaaagt cgaagggggc ttttattttg gtttgatgtt | 2940 |
| gcgattaata gcaatacgat tgcaataaac aaaaggatcc atgcaagcgg caactgttgt | 3000 |
| gattaaccgc cgcgctctgc gacacaacct gcaacgtctt cgtgaactgg cccctgccag | 3060 |
| taaaatggtt gcggtggtga aagcgaacgc ttatggtcac ggtcttcttg agaccgcgcg | 3120 |
| aacgctcccc gatgctgacg cctttggcgt agcccgtctc gaagaagctc tgcgactgcg | 3180 |
| tgcgggggga atcaccaaac ctgtactgtt actcgaaggc ttttttgatg ccagagatct | 3240 |
| gccgacgatt tctgcgcaac attttcatac cgccgtgcat aacgaagaac agctggctgc | 3300 |
| gctggaagag gctagcctgg acgagccggt taccgtctgg atgaaactcg ataccggtat | 3360 |
| gcaccgtctg ggcgtaaggc cggaacaggc tgaggcgttt tatcatcgcc tgacccagtg | 3420 |
| caaaaacgtt cgtcagccgg tgaatatcgt cagccatttt gcgcgcgcgg atgaaccaaa | 3480 |
| atgtggcgca accgagaaac aactcgctat ctttaatacc ttttgcgaag gcaaacctgg | 3540 |
| tcaacgttcc attgccgcgt cgggtggcat tctgctgtgg ccacagtcgc attttgactg | 3600 |
| ggtgcgcccg ggcatcattc tttatggcgt ctcgccgctg gaagatcgct ccaccggtgc | 3660 |
| cgattttggc tgtcagccag tgatgtcact aacctccagc ctgattgccg tgcgtgagca | 3720 |
| taaagccgga gagcctgttg gttatggtgg aacctgggta agcgaacgtg atacccgtct | 3780 |
| tggcgtagtc gcgatgggct atggcgatgg ttatccgcgc gccgcgccgt ccggtacgcc | 3840 |
| agtgctggtg aacggtcgcg aagtaccgat tgtcgggcgc gtggcgatgg atatgatctg | 3900 |
| cgtagactta ggtccacagg cgcaggacaa agccggggat ccggtcattt tatggggcga | 3960 |
| aggtttgccc gtagaacgta tcgctgaaat gacgaaagta agcgcttacg aacttattac | 4020 |
| gcgcctgact tcaagggtcg cgatgaaata cgtggattaa acacgttact aaagggaatg | 4080 |
| gagaccgggg cccttcaata gagttcttaa cgttaatccg aaaaaaacta acgttaatat | 4140 |
| taaaaaataa gatccgcttg tgaattatgt ataatttgat tagactaaag aataggagaa | 4200 |
| agtatgatga tatttaaaaa actttctcgt taagataggt tgttggtgag catgttatat | 4260 |
| acggatgtat cggtttcctt aatgcaaaat tttgttgcta tcttattaat ttttctatta | 4320 |
| tatagatata ttcaaagaaa gataacattt aaacggatca tattagatat tttaatagcg | 4380 |
| attatttttt caatattata tctgtttatt tcagatgcgt cattacttgt aatggtatta | 4440 |
| atgcgattag ggtggcattt tcatcaacaa aaagaaaata agataaaaac gactgataca | 4500 |
| gctaatttaa ttctaattat cgtgatccag ttattgttag ttgcggttgg gactattatt | 4560 |
| agtcagttta ccatatcgat tatcaaaagt gatttcagcc aaaatatatt gaacaatagt | 4620 |
| gcaacagata taactttatt aggtattttc tttgctgttt tatttgacgg cttgttcttt | 4680 |
| atattattga agaataagcg gactgaatta caacatttaa atcaagaaat cattgaattt | 4740 |
| tcgttagaaa aacaatattt tatatttata tttattttat ttatagtaat agaaattatt | 4800 |
| ttagcagttg ggaatcttca aggagtaaca gccacgatat tattaaccat tatcattatt | 4860 |
| ttttgtgtcc ttatcgggat gactttttgg caagtgatgc tttttttgaa ggcttattcg | 4920 |
| attcgccaag aagccaatga ccaattggtc cggaatcaac aacttcaaga ttatctagtc | 4980 |
| aatatcgaac agcagtacac cgaattacgg cgatttaagc atgattatca aaacatctta | 5040 |
| ttatcgttgg agagttttgc cgaaaagggc gatcagcaac agtttaaggc gtattaccaa | 5100 |
| gaattattag cacaacggcc aattcaaagt gaaatccaag gggcagtcat tgcacaactc | 5160 |
| gactacttga aaaatgatcc tattcgagga ttagtcattc aaaagttttt ggcagccaaa | 5220 |
| caggctggtg ttactttaaa attcgaaatg accgaaccaa tcgaattagc aaccgctaat | 5280 |
| ctattaacgg ttattcggat tatcggtatt ttattagaca atgcgattga acaagccgtt | 5340 |
| caagaaaccg atcaattggt gagttgtgct ttcttacaat ctgatggttt aatcgaaatt | 5400 |
| acgattgaaa atacggccag tcaagttaag aatctccaag cattttcaga gttaggctat | 5460 |
| tcaacgaaag gcgctggtcg ggggactggt ttagctaatg tgcaggattt gattgccaaa | 5520 |
| caaaccaatt tattcttaga aacacagatt gaaaatagaa agttacgaca gacattgatg | 5580 |
| attacggagg aaacttaatt tgtatcccgt ttatttatta gaggatgatt tacagcaaca | 5640 |
| agcgatttat cagcaaatta tcgcgaatac gattatgatt aacgaatttg caatgacttt | 5700 |
| aacatgcgct gccagtgata ctgagacatt gttggcggca attaaggatc agcaacgagg | 5760 |
| tttattcttt ttggatatgg aaattgagga taaccgccaa gccggtttag aagtggcaac | 5820 |
| taagattcgg cagatgatgc cgtttgcgca aattgtcttc attacaaccc acgaggaact | 5880 |
| gacattatta acgttagaac gaaaaatagc gcctttagat tacattctca aggaccaaac | 5940 |
| aatggctgaa atcaaaaggc aattgattga tgatctattg ttagctgaga agcaaaacga | 6000 |
| ggcggcagcg tatcaccgag aaaatttatt tagttataaa ataggtcctc gctttttctc | 6060 |
| attaccatta aaggaagttg tttatttata tactgaaaaa gaaaatccgg gtcatattaa | 6120 |
| tttgttagcc gttaccagaa aggttacttt tccaggaaat ttaaatgcgc tggaagccca | 6180 |
| atatccaatg ctctttcggt gtgataaaag ttacttagtt aacctatcta atattgccaa | 6240 |
| ttatgacagt aaaacacgga gtttaaaatt tgtagatggc agtgaggcaa aagtctcgtt | 6300 |
| ccggaaatca cgggaactag tggccaaatt aaaacaaatg atgtagcgcc tgcagcacgc | 6360 |
| caaatgatcc cagtaaaaag ccacccgcat ggcgggtggc tttttattag ccctagaagg | 6420 |
| gcttcccaca cgcatttcag cgccttagtg ccttagtttg tgaatcatag gtggtatagt | 6480 |
| cccgaaatac ccgtctaagg aattgtcaga taggcctaat gactggcttt tataatatga | 6540 |
| gataatgccg actgtacttt ttacagtcgg ttttctaatg tcactaacct gccccgttag | 6600 |
| ttgaagaagg tttttatatt acagctccag atctaccggt gggcccatat taacgtttaa | 6660 |
| ccgataaagt tgaacgttaa tatttttttt gcgcagaaat ggtaaattga agcataatag | 6720 |
| tcttgtaagg tatttagctg gctggcgtaa agtatgcttt ataaaataat atataggagt | 6780 |
| atgattc | 6787 |
| human aldehyde dehydrogenase 1B1 (UNIPROT SEQ: P30837; SEQ ID NO: 10): | |
| M L R F L A P R L L S L Q G R T A R Y S S A A A L | |
| P S P I L N P D I P Y N Q L F I N N E W Q D A V S | |
| K K T F P T V N P T T G E V I G H V A E G D R A D | |
| V D R A V K A A R E A F R L G S P W R R M D A S E | |
| R G R L L N L L A D L V E R D R V Y L A S L E T L | |
| D N G K P F Q E S Y A L D L D E V I K V Y R Y F A | |
| G W A D K W H G K T I P M D G Q H F C F T R H E P | |
| V G V C G Q I I P W N F P L V M Q G W K L A P A L | |
| A T G N T V V M K V A E Q T P L S A L Y L A S L I | |
| K E A G F P P G V V N I I T G Y G P T A G A A I A | |
| Q H V D V D K V A F T G S T E V G H L I Q K A A G | |
| D S N L K R V T L E L G G K S P S I V L A D A D M | |
| E H A V E Q C H E A L F F N M G Q C C C A G S R T | |
| F V E E S I Y N E F L E R T V E K A K Q R K V G N | |
| P F E L D T Q Q G P Q V D K E Q F E R V L G Y I Q | |
| L G Q K E G A K L L C G G E R F G E R G F F I K P | |
| T V F G G V Q D D M R I A K E E I F G P V Q P L F | |
| K F K K I E E V V E R A N N T R Y G L A A A V F T | |
| R D L D K A M Y F T Q A L Q A G T V W V N T Y N I | |
| V T C H T P F G G F K E S G N G R E L G E D G L K | |
| A Y T E V K T V T I K V P Q K N S | |
| p3050alarAmuc_1100_alcA-al1b1-sh71 | |
| (SEQ ID NO: 11) | |
| atatgaaaaa atttaacttt aaaaccatgt tgctattagt tttggctagt tgtgtcttcg | 60 |
| gggtcgtcgt taacgtgact actagtcttg gaccacaaac cgcaatcacc gcccaggcct | 120 |
| ccaaaggagg tatcgtcaat tccaaacgca gtgaactgga caaaaaaatc agcatcgccg | 180 |
| ccaaggaaat caagtccgcc aatgctgcgg aaatcactcc gagccgatca tccaacgaag | 240 |
| agctggaaaa agaactgaac cgctatgcca aggccgtggg cagcctggaa acggcctaca | 300 |
| agcccttcct tgcctcctcc gcgctggtcc ccaccacgcc cacggcattc cagaatgaac | 360 |
| tgaaaacatt cagggattcc ctgatctcct cctgcaagaa aaagaacatt ctcataacgg | 420 |
| acacatcctc ctggctcggt ttccaggttt acagcaccca ggctccctct gttcaggcgg | 480 |
| cctccacgct gggttttgaa ttgaaagcca tcaacagcct ggtcaacaaa ctggcggaat | 540 |
| gcggcctgtc caaattcatc aaggtgtacc gcccccagct ccccattgaa accccggcga | 600 |
| acaatccgga agaatcggac gaagccgacc aggccccatg gactcccatg cctctggaaa | 660 |
| tagccttcca gggcgaccgg gaaagtgtat tgaaagccat gaacgccata accggcatgc | 720 |
| aggactatct gttcacggtc aactccatcc gtatccgcaa cgaacggatg atgccccctc | 780 |
| ccatcgccaa tccggcagcc gccaaacctg ccgcggccca acccgccacg ggtgcggctt | 840 |
| ccctgactcc ggcggatgag gcggctgcac ctgcagcccc ggccatccag caagtcatca | 900 |
| agccttacat gggcaaggag caggtctttg tccaggtctc cctgaatctg gtccacttca | 960 |
| accagcccaa ggctcaggaa ccgtctgaag attaatactt gaaaaaaaaa aaccccgccc | 1020 |
| ctgacagggc ggggtttttt tttccattgt ggtgatcgtt ccgacatgct tgtctgcatg | 1080 |
| ggtttctgcg tgtcgggact caagtgatct ggggcttgat gcatgtggga cagcacgagg | 1140 |
| tagaggtgga aactgacata cgactccgtt acatgccccg tttaagcgct atgcgtatcg | 1200 |
| tgccgtctaa tcccgtgatg gagcgttatc aggcacagta cggactggat gccctcatgg | 1260 |
| cgaaccacaa acctcaggag ctccctacgt actgagctat ccgcgcattg cttcgcctca | 1320 |
| tagctaaacg ggcatgacac acaatccgac catactcagg aaaacgcttc cactgtacaa | 1380 |
| agaggtccac ttcatctgga gaggccctag gaggtatgct cagattcttg gcgcctcgcc | 1440 |
| ttcttagcct ccaaggacgt acagccagat attcaagtgc agcagctctt ccgagcccga | 1500 |
| ttctcaatcc ggatattccg tataaccaac tgttcattaa caacgagtgg caagacgcag | 1560 |
| taagcaagaa aacgtttccg acagtcaatc caactaccgg agaagtgatc ggccacgttg | 1620 |
| cagaaggtga tcgggccgat gtcgatcgtg cagttaaagc tgcgagagag gctttcaggc | 1680 |
| ttgggtcccc atggcggagg atggatgctt cggaacgtgg cagactgctc aatctgttag | 1740 |
| ctgatcttgt agagcgagat cgggtatatc tggcatctct ggaaacactg gacaatggga | 1800 |
| agccatttca ggaatcctat gcccttgatc tggatgaggt gattaaggtg tatcgctatt | 1860 |
| ttgctggctg ggcagataag tggcatggga aaacaatacc gatggacggc cagcactttt | 1920 |
| gctttaccag acatgaacct gttggagtat gtggtcaaat cataccctgg aactttccgc | 1980 |
| tggtaatgca aggctggaaa ttagcacccg cgttagcgac gggtaataca gtggtcatga | 2040 |
| aagtagctga gcaaacgccg ctttcagcct tgtatttagc ctctcttatc aaagaagctg | 2100 |
| gatttcctcc gggtgttgtt aacatcatta caggatacgg ccctacagct ggcgcggcaa | 2160 |
| tcgcgcaaca tgtggacgta gacaaagtcg cctttactgg ctcaaccgaa gtcgggcatc | 2220 |
| tgatccagaa agctgctggc gatagcaact tgaaacgcgt tacactggag ttaggaggaa | 2280 |
| aatctccgag tattgtctta gcggatgcag atatggaaca tgctgttgaa cagtgccatg | 2340 |
| aagccttatt cttcaacatg ggtcagtgct gttgtgcggg atctcgtacc tttgtggaag | 2400 |
| agtccattta caatgaattt ctggaacgta ccgttgagaa ggcgaaacaa cgcaaagtcg | 2460 |
| gaaatccgtt tgagctggac acgcaacaag gtccacaagt ggacaaagaa cagtttgaaa | 2520 |
| gagttttggg ctacattcag ctcggacaga aagaaggagc caagttactt tgcggaggcg | 2580 |
| aacgatttgg tgaacggggt ttcttcatca aaccaactgt ctttggtgga gtgcaggatg | 2640 |
| acatgaggat tgcgaaagaa gagattttcg gccctgtgca acctctgttc aaatttaaga | 2700 |
| aaatcgaaga agttgtggaa agagccaaca atacgcggta tggccttgcg gcggcagtct | 2760 |
| ttactcgcga tttagacaag gcgatgtact ttacgcaagc cttgcaggca gggacagttt | 2820 |
| gggtgaatac gtataacatt gttacatgtc acacaccttt tggaggcttt aaagagtcag | 2880 |
| ggaatggacg agaattgggc gaagatgggt tgaaagcata cactgaggtc aaaacagtca | 2940 |
| cgataaaagt accccagaag aattcgtaat acttgaaaaa aaaaaacccc gcccctgaca | 3000 |
| gggcggggtt ttttttcatg gatcgatttt ttattaaaac gtctcaaaat cgtttctgag | 3060 |
| acgttttagc gtttatttcg tttagttatc ggcataatcg ttaaaacagg cgttatcgta | 3120 |
| gcgtaaaagc ccttgagcgt agcgtgcttt gcagcgaaga tgttgtctgt tagattatga | 3180 |
| aagccgatga ctgaatgaaa taataagcgc agcgtccttc tatttcggtt ggaggaggct | 3240 |
| caagggagtt tgagggaatg aaattccctc atgggtttga ttttaaaaat tgcttgcaat | 3300 |
| tttgccgagc ggtagcgctg gaaaaatttt tgaaaaaaat ttggaatttg gaaaaaaatg | 3360 |
| gggggaaagg aagcgaattt tgcttccgta ctacgacccc ccattaagtg ccgagtgcca | 3420 |
| atttttgtgc caaaaacgct ctatcccaac tggctcaagg gtttgagggg tttttcaatc | 3480 |
| gccaacgaat cgccaacgtt ttcgccaacg ttttttataa atctatattt aagtagcttt | 3540 |
| attgttgttt ttatgattac aaagtgatac actaatttta taaaattatt tgattggagt | 3600 |
| tttttaaatg gtgatttcag aatcgaaaaa aagagttatg atttctctga caaaagagca | 3660 |
| agataaaaaa ttaacagata tggcgaaaca aaaaggtttt tcaaaatctg cggttgcggc | 3720 |
| gttagctata gaagaatatg caagaaagga atcagaataa aaaaaataag cgaaagctcg | 3780 |
| cgtttttaga aggatacgag ttttcgctac ttgtttttga taaggtaata tatcatggct | 3840 |
| attaaatact aaagctagaa attttggatt tttattatat cctgactcaa ttcctaatga | 3900 |
| ttggaaagaa aaattagaga gtttgggcgt atctatggct gtcagtcctt tacacgatat | 3960 |
| ggacgaaaaa aaagataaag atacatggaa tagtagtgat gttatacgaa atggaaagca | 4020 |
| ctataaaaaa ccacactatc acgttatata tattgcacga aatcctgtaa caatagaaag | 4080 |
| cgttaggaac aagattaagc gaaaattggg gaatagttca gttgctcatg ttgagatact | 4140 |
| tgattatatc aaaggttcat atgaatattt gactcatgaa tcaaaggacg ctattgctaa | 4200 |
| gaataaacat atatacgaca aaaaagatat tttgaacatt aatgattttg atattgaccg | 4260 |
| ctatataaca cttgatgaaa gccaaaaaag agaattgaag aatttacttt tagatatagt | 4320 |
| ggatgactat aatttggtaa atacaaaaga tttaatggct tttattcgcc ttaggggagc | 4380 |
| ggagtttgga attttaaata cgaatgatgt aaaagatatt gtttcaacaa actctagcgc | 4440 |
| ctttagatta tggtttgagg gcaattatca gtgtggatat agagcaagtt atgcaaaggt | 4500 |
| tcttgatgct gaaacggggg aaataaaatg acaaacaaag aaaaagagtt atttgctgaa | 4560 |
| aatgaggaat taaaaaaaga aattaaggac ttaaaagagc gtattgaaag atacagagaa | 4620 |
| atggaagttg aattaagtac aacaatagat ttattgagag gagggattat tgaataaata | 4680 |
| aaagcccccc tgacgaaagt cgaagggggc ttttattttg gtttgatgtt gcgattaata | 4740 |
| gcaatacgat tgcaataaac aaaaggatcc atgcaagcgg caactgttgt gattaaccgc | 4800 |
| cgcgctctgc gacacaacct gcaacgtctt cgtgaactgg cccctgccag taaaatggtt | 4860 |
| gcggtggtga aagcgaacgc ttatggtcac ggtcttcttg agaccgcgcg aacgctcccc | 4920 |
| gatgctgacg cctttggcgt agcccgtctc gaagaagctc tgcgactgcg tgcgggggga | 4980 |
| atcaccaaac ctgtactgtt actcgaaggc ttttttgatg ccagagatct gccgacgatt | 5040 |
| tctgcgcaac attttcatac cgccgtgcat aacgaagaac agctggctgc gctggaagag | 5100 |
| gctagcctgg acgagccggt taccgtctgg atgaaactcg ataccggtat gcaccgtctg | 5160 |
| ggcgtaaggc cggaacaggc tgaggcgttt tatcatcgcc tgacccagtg caaaaacgtt | 5220 |
| cgtcagccgg tgaatatcgt cagccatttt gcgcgcgcgg atgaaccaaa atgtggcgca | 5280 |
| accgagaaac aactcgctat ctttaatacc ttttgcgaag gcaaacctgg tcaacgttcc | 5340 |
| attgccgcgt cgggtggcat tctgctgtgg ccacagtcgc attttgactg ggtgcgcccg | 5400 |
| ggcatcattc tttatggcgt ctcgccgctg gaagatcgct ccaccggtgc cgattttggc | 5460 |
| tgtcagccag tgatgtcact aacctccagc ctgattgccg tgcgtgagca taaagccgga | 5520 |
| gagcctgttg gttatggtgg aacctgggta agcgaacgtg atacccgtct tggcgtagtc | 5580 |
| gcgatgggct atggcgatgg ttatccgcgc gccgcgccgt ccggtacgcc agtgctggtg | 5640 |
| aacggtcgcg aagtaccgat tgtcgggcgc gtggcgatgg atatgatctg cgtagactta | 5700 |
| ggtccacagg cgcaggacaa agccggggat ccggtcattt tatggggcga aggtttgccc | 5760 |
| gtagaacgta tcgctgaaat gacgaaagta agcgcttacg aacttattac gcgcctgact | 5820 |
| tcaagggtcg cgatgaaata cgtggattaa acacgttact aaagggaatg gagaccgggg | 5880 |
| cccttcaata gagttcttaa cgttaatccg aaaaaaacta acgttaatat taaaaaataa | 5940 |
| gatccgcttg tgaattatgt ataatttgat tagactaaag aataggagaa agtatgatga | 6000 |
| tatttaaaaa actttctcgt taagataggt tgttggtgag catgttatat acggatgtat | 6060 |
| cggtttcctt aatgcaaaat tttgttgcta tcttattaat ttttctatta tatagatata | 6120 |
| ttcaaagaaa gataacattt aaacggatca tattagatat tttaatagcg attatttttt | 6180 |
| caatattata tctgtttatt tcagatgcgt cattacttgt aatggtatta atgcgattag | 6240 |
| ggtggcattt tcatcaacaa aaagaaaata agataaaaac gactgataca gctaatttaa | 6300 |
| ttctaattat cgtgatccag ttattgttag ttgcggttgg gactattatt agtcagttta | 6360 |
| ccatatcgat tatcaaaagt gatttcagcc aaaatatatt gaacaatagt gcaacagata | 6420 |
| taactttatt aggtattttc tttgctgttt tatttgacgg cttgttcttt atattattga | 6480 |
| agaataagcg gactgaatta caacatttaa atcaagaaat cattgaattt tcgttagaaa | 6540 |
| aacaatattt tatatttata tttattttat ttatagtaat agaaattatt ttagcagttg | 6600 |
| ggaatcttca aggagtaaca gccacgatat tattaaccat tatcattatt ttttgtgtcc | 6660 |
| ttatcgggat gactttttgg caagtgatgc tttttttgaa ggcttattcg attcgccaag | 6720 |
| aagccaatga ccaattggtc cggaatcaac aacttcaaga ttatctagtc aatatcgaac | 6780 |
| agcagtacac cgaattacgg cgatttaagc atgattatca aaacatctta ttatcgttgg | 6840 |
| agagttttgc cgaaaagggc gatcagcaac agtttaaggc gtattaccaa gaattattag | 6900 |
| cacaacggcc aattcaaagt gaaatccaag gggcagtcat tgcacaactc gactacttga | 6960 |
| aaaatgatcc tattcgagga ttagtcattc aaaagttttt ggcagccaaa caggctggtg | 7020 |
| ttactttaaa attcgaaatg accgaaccaa tcgaattagc aaccgctaat ctattaacgg | 7080 |
| ttattcggat tatcggtatt ttattagaca atgcgattga acaagccgtt caagaaaccg | 7140 |
| atcaattggt gagttgtgct ttcttacaat ctgatggttt aatcgaaatt acgattgaaa | 7200 |
| atacggccag tcaagttaag aatctccaag cattttcaga gttaggctat tcaacgaaag | 7260 |
| gcgctggtcg ggggactggt ttagctaatg tgcaggattt gattgccaaa caaaccaatt | 7320 |
| tattcttaga aacacagatt gaaaatagaa agttacgaca gacattgatg attacggagg | 7380 |
| aaacttaatt tgtatcccgt ttatttatta gaggatgatt tacagcaaca agcgatttat | 7440 |
| cagcaaatta tcgcgaatac gattatgatt aacgaatttg caatgacttt aacatgcgct | 7500 |
| gccagtgata ctgagacatt gttggcggca attaaggatc agcaacgagg tttattcttt | 7560 |
| ttggatatgg aaattgagga taaccgccaa gccggtttag aagtggcaac taagattcgg | 7620 |
| cagatgatgc cgtttgcgca aattgtcttc attacaaccc acgaggaact gacattatta | 7680 |
| acgttagaac gaaaaatagc gcctttagat tacattctca aggaccaaac aatggctgaa | 7740 |
| atcaaaaggc aattgattga tgatctattg ttagctgaga agcaaaacga ggcggcagcg | 7800 |
| tatcaccgag aaaatttatt tagttataaa ataggtcctc gctttttctc attaccatta | 7860 |
| aaggaagttg tttatttata tactgaaaaa gaaaatccgg gtcatattaa tttgttagcc | 7920 |
| gttaccagaa aggttacttt tccaggaaat ttaaatgcgc tggaagccca atatccaatg | 7980 |
| ctctttcggt gtgataaaag ttacttagtt aacctatcta atattgccaa ttatgacagt | 8040 |
| aaaacacgga gtttaaaatt tgtagatggc agtgaggcaa aagtctcgtt ccggaaatca | 8100 |
| cgggaactag tggccaaatt aaaacaaatg atgtagcgcc tgcagcacgc caaatgatcc | 8160 |
| cagtaaaaag ccacccgcat ggcgggtggc tttttattag ccctagaagg gcttcccaca | 8220 |
| cgcatttcag cgccttagtg ccttagtttg tgaatcatag gtggtatagt cccgaaatac | 8280 |
| ccgtctaagg aattgtcaga taggcctaat gactggcttt tataatatga gataatgccg | 8340 |
| actgtacttt ttacagtcgg ttttctaatg tcactaacct gccccgttag ttgaagaagg | 8400 |
| tttttatatt acagctccag atctaccggt gggcccatat taacgtttaa ccgataaagt | 8460 |
| tgaacgttaa tatttttttt gcgcagaaat ggtaaattga agcataatag tcttgtaagg | 8520 |
| tatttagctg gctggcgtaa agtatgcttt ataaaataat atataggagt atgattc | 8577 |
| Terminator iGEM-part BBa_B1006 | |
| (SEQ ID NO: 12) | |
| aaaaaaaaac cccgcccctg acagggcggg gtttttttt | |
| 5′UTR | |
| (SEQ ID NO: 13) | |
| AGGAGGT | |
| 3′UTR | |
| (SEQ ID NO: 14) | |
| TACTTGAA | |
| p3050Alr_Amuc1100_sh71 with 5′UTR, 3′UTR and terminator | |
| (SEQ ID NO: 15) | |
| atatgaaaaa atttaacttt aaaaccatgt tgctattagt tttggctagt tgtgtcttcg | 60 |
| gggtcgtcgt taacgtgact actagtcttg gaccacaaac cgcaatcacc gcccaggcct | 120 |
| ccaaaggagg tatcgtcaat tccaaacgca gtgaactgga caaaaaaatc agcatcgccg | 180 |
| ccaaggaaat caagtccgcc aatgctgcgg aaatcactcc gagccgatca tccaacgaag | 240 |
| agctggaaaa agaactgaac cgctatgcca aggccgtggg cagcctggaa acggcctaca | 300 |
| agcccttcct tgcctcctcc gcgctggtcc ccaccacgcc cacggcattc cagaatgaac | 360 |
| tgaaaacatt cagggattcc ctgatctcct cctgcaagaa aaagaacatt ctcataacgg | 420 |
| acacatcctc ctggctcggt ttccaggttt acagcaccca ggctccctct gttcaggcgg | 480 |
| cctccacgct gggttttgaa ttgaaagcca tcaacagcct ggtcaacaaa ctggcggaat | 540 |
| gcggcctgtc caaattcatc aaggtgtacc gcccccagct ccccattgaa accccggcga | 600 |
| acaatccgga agaatcggac gaagccgacc aggccccatg gactcccatg cctctggaaa | 660 |
| tagccttcca gggcgaccgg gaaagtgtat tgaaagccat gaacgccata accggcatgc | 720 |
| aggactatct gttcacggtc aactccatcc gtatccgcaa cgaacggatg atgccccctc | 780 |
| ccatcgccaa tccggcagcc gccaaacctg ccgcggccca acccgccacg ggtgcggctt | 840 |
| ccctgactcc ggcggatgag gcggctgcac ctgcagcccc ggccatccag caagtcatca | 900 |
| agccttacat gggcaaggag caggtctttg tccaggtctc cctgaatctg gtccacttca | 960 |
| accagcccaa ggctcaggaa ccgtctgaag attaatactt gaaaaaaaaa aaccccgccc | 1020 |
| ctgacagggc ggggtttttt ttcatggatc gattttttat taaaacgtct caaaatcgtt | 1080 |
| tctgagacgt tttagcgttt atttcgttta gttatcggca taatcgttaa aacaggcgtt | 1140 |
| atcgtagcgt aaaagccctt gagcgtagcg tgctttgcag cgaagatgtt gtctgttaga | 1200 |
| ttatgaaagc cgatgactga atgaaataat aagcgcagcg tccttctatt tcggttggag | 1260 |
| gaggctcaag ggagtttgag ggaatgaaat tccctcatgg gtttgatttt aaaaattgct | 1320 |
| tgcaattttg ccgagcggta gcgctggaaa aatttttgaa aaaaatttgg aatttggaaa | 1380 |
| aaaatggggg gaaaggaagc gaattttgct tccgtactac gaccccccat taagtgccga | 1440 |
| gtgccaattt ttgtgccaaa aacgctctat cccaactggc tcaagggttt gaggggtttt | 1500 |
| tcaatcgcca acgaatcgcc aacgttttcg ccaacgtttt ttataaatct atatttaagt | 1560 |
| agctttattg ttgtttttat gattacaaag tgatacacta attttataaa attatttgat | 1620 |
| tggagttttt taaatggtga tttcagaatc gaaaaaaaga gttatgattt ctctgacaaa | 1680 |
| agagcaagat aaaaaattaa cagatatggc gaaacaaaaa ggtttttcaa aatctgcggt | 1740 |
| tgcggcgtta gctatagaag aatatgcaag aaaggaatca gaataaaaaa aataagcgaa | 1800 |
| agctcgcgtt tttagaagga tacgagtttt cgctacttgt ttttgataag gtaatatatc | 1860 |
| atggctatta aatactaaag ctagaaattt tggattttta ttatatcctg actcaattcc | 1920 |
| taatgattgg aaagaaaaat tagagagttt gggcgtatct atggctgtca gtcctttaca | 1980 |
| cgatatggac gaaaaaaaag ataaagatac atggaatagt agtgatgtta tacgaaatgg | 2040 |
| aaagcactat aaaaaaccac actatcacgt tatatatatt gcacgaaatc ctgtaacaat | 2100 |
| agaaagcgtt aggaacaaga ttaagcgaaa attggggaat agttcagttg ctcatgttga | 2160 |
| gatacttgat tatatcaaag gttcatatga atatttgact catgaatcaa aggacgctat | 2220 |
| tgctaagaat aaacatatat acgacaaaaa agatattttg aacattaatg attttgatat | 2280 |
| tgaccgctat ataacacttg atgaaagcca aaaaagagaa ttgaagaatt tacttttaga | 2340 |
| tatagtggat gactataatt tggtaaatac aaaagattta atggctttta ttcgccttag | 2400 |
| gggagcggag tttggaattt taaatacgaa tgatgtaaaa gatattgttt caacaaactc | 2460 |
| tagcgccttt agattatggt ttgagggcaa ttatcagtgt ggatatagag caagttatgc | 2520 |
| aaaggttctt gatgctgaaa cgggggaaat aaaatgacaa acaaagaaaa agagttattt | 2580 |
| gctgaaaatg aggaattaaa aaaagaaatt aaggacttaa aagagcgtat tgaaagatac | 2640 |
| agagaaatgg aagttgaatt aagtacaaca atagatttat tgagaggagg gattattgaa | 2700 |
| taaataaaag cccccctgac gaaagtcgaa gggggctttt attttggttt gatgttgcga | 2760 |
| ttaatagcaa tacgattgca ataaacaaaa ggatccatgc aagcggcaac tgttgtgatt | 2820 |
| aaccgccgcg ctctgcgaca caacctgcaa cgtcttcgtg aactggcccc tgccagtaaa | 2880 |
| atggttgcgg tggtgaaagc gaacgcttat ggtcacggtc ttcttgagac cgcgcgaacg | 2940 |
| ctccccgatg ctgacgcctt tggcgtagcc cgtctcgaag aagctctgcg actgcgtgcg | 3000 |
| gggggaatca ccaaacctgt actgttactc gaaggctttt ttgatgccag agatctgccg | 3060 |
| acgatttctg cgcaacattt tcataccgcc gtgcataacg aagaacagct ggctgcgctg | 3120 |
| gaagaggcta gcctggacga gccggttacc gtctggatga aactcgatac cggtatgcac | 3180 |
| cgtctgggcg taaggccgga acaggctgag gcgttttatc atcgcctgac ccagtgcaaa | 3240 |
| aacgttcgtc agccggtgaa tatcgtcagc cattttgcgc gcgcggatga accaaaatgt | 3300 |
| ggcgcaaccg agaaacaact cgctatcttt aatacctttt gcgaaggcaa acctggtcaa | 3360 |
| cgttccattg ccgcgtcggg tggcattctg ctgtggccac agtcgcattt tgactgggtg | 3420 |
| cgcccgggca tcattcttta tggcgtctcg ccgctggaag atcgctccac cggtgccgat | 3480 |
| tttggctgtc agccagtgat gtcactaacc tccagcctga ttgccgtgcg tgagcataaa | 3540 |
| gccggagagc ctgttggtta tggtggaacc tgggtaagcg aacgtgatac ccgtcttggc | 3600 |
| gtagtcgcga tgggctatgg cgatggttat ccgcgcgccg cgccgtccgg tacgccagtg | 3660 |
| ctggtgaacg gtcgcgaagt accgattgtc gggcgcgtgg cgatggatat gatctgcgta | 3720 |
| gacttaggtc cacaggcgca ggacaaagcc ggggatccgg tcattttatg gggcgaaggt | 3780 |
| ttgcccgtag aacgtatcgc tgaaatgacg aaagtaagcg cttacgaact tattacgcgc | 3840 |
| ctgacttcaa gggtcgcgat gaaatacgtg gattaaacac gttactaaag ggaatggaga | 3900 |
| ccggggccct tcaatagagt tcttaacgtt aatccgaaaa aaactaacgt taatattaaa | 3960 |
| aaataagatc cgcttgtgaa ttatgtataa tttgattaga ctaaagaata ggagaaagta | 4020 |
| tgatgatatt taaaaaactt tctcgttaag ataggttgtt ggtgagcatg ttatatacgg | 4080 |
| atgtatcggt ttccttaatg caaaattttg ttgctatctt attaattttt ctattatata | 4140 |
| gatatattca aagaaagata acatttaaac ggatcatatt agatatttta atagcgatta | 4200 |
| ttttttcaat attatatctg tttatttcag atgcgtcatt acttgtaatg gtattaatgc | 4260 |
| gattagggtg gcattttcat caacaaaaag aaaataagat aaaaacgact gatacagcta | 4320 |
| atttaattct aattatcgtg atccagttat tgttagttgc ggttgggact attattagtc | 4380 |
| agtttaccat atcgattatc aaaagtgatt tcagccaaaa tatattgaac aatagtgcaa | 4440 |
| cagatataac tttattaggt attttctttg ctgttttatt tgacggcttg ttctttatat | 4500 |
| tattgaagaa taagcggact gaattacaac atttaaatca agaaatcatt gaattttcgt | 4560 |
| tagaaaaaca atattttata tttatattta ttttatttat agtaatagaa attattttag | 4620 |
| cagttgggaa tcttcaagga gtaacagcca cgatattatt aaccattatc attatttttt | 4680 |
| gtgtccttat cgggatgact ttttggcaag tgatgctttt tttgaaggct tattcgattc | 4740 |
| gccaagaagc caatgaccaa ttggtccgga atcaacaact tcaagattat ctagtcaata | 4800 |
| tcgaacagca gtacaccgaa ttacggcgat ttaagcatga ttatcaaaac atcttattat | 4860 |
| cgttggagag ttttgccgaa aagggcgatc agcaacagtt taaggcgtat taccaagaat | 4920 |
| tattagcaca acggccaatt caaagtgaaa tccaaggggc agtcattgca caactcgact | 4980 |
| acttgaaaaa tgatcctatt cgaggattag tcattcaaaa gtttttggca gccaaacagg | 5040 |
| ctggtgttac tttaaaattc gaaatgaccg aaccaatcga attagcaacc gctaatctat | 5100 |
| taacggttat tcggattatc ggtattttat tagacaatgc gattgaacaa gccgttcaag | 5160 |
| aaaccgatca attggtgagt tgtgctttct tacaatctga tggtttaatc gaaattacga | 5220 |
| ttgaaaatac ggccagtcaa gttaagaatc tccaagcatt ttcagagtta ggctattcaa | 5280 |
| cgaaaggcgc tggtcggggg actggtttag ctaatgtgca ggatttgatt gccaaacaaa | 5340 |
| ccaatttatt cttagaaaca cagattgaaa atagaaagtt acgacagaca ttgatgatta | 5400 |
| cggaggaaac ttaatttgta tcccgtttat ttattagagg atgatttaca gcaacaagcg | 5460 |
| atttatcagc aaattatcgc gaatacgatt atgattaacg aatttgcaat gactttaaca | 5520 |
| tgcgctgcca gtgatactga gacattgttg gcggcaatta aggatcagca acgaggttta | 5580 |
| ttctttttgg atatggaaat tgaggataac cgccaagccg gtttagaagt ggcaactaag | 5640 |
| attcggcaga tgatgccgtt tgcgcaaatt gtcttcatta caacccacga ggaactgaca | 5700 |
| ttattaacgt tagaacgaaa aatagcgcct ttagattaca ttctcaagga ccaaacaatg | 5760 |
| gctgaaatca aaaggcaatt gattgatgat ctattgttag ctgagaagca aaacgaggcg | 5820 |
| gcagcgtatc accgagaaaa tttatttagt tataaaatag gtcctcgctt tttctcatta | 5880 |
| ccattaaagg aagttgttta tttatatact gaaaaagaaa atccgggtca tattaatttg | 5940 |
| ttagccgtta ccagaaaggt tacttttcca ggaaatttaa atgcgctgga agcccaatat | 6000 |
| ccaatgctct ttcggtgtga taaaagttac ttagttaacc tatctaatat tgccaattat | 6060 |
| gacagtaaaa cacggagttt aaaatttgta gatggcagtg aggcaaaagt ctcgttccgg | 6120 |
| aaatcacggg aactagtggc caaattaaaa caaatgatgt agcgcctgca gcacgccaaa | 6180 |
| tgatcccagt aaaaagccac ccgcatggcg ggtggctttt tattagccct agaagggctt | 6240 |
| cccacacgca tttcagcgcc ttagtgcctt agtttgtgaa tcataggtgg tatagtcccg | 6300 |
| aaatacccgt ctaaggaatt gtcagatagg cctaatgact ggcttttata atatgagata | 6360 |
| atgccgactg tactttttac agtcggtttt ctaatgtcac taacctgccc cgttagttga | 6420 |
| agaaggtttt tatattacag ctccagatct accggtgggc ccatattaac gtttaaccga | 6480 |
| taaagttgaa cgttaatatt ttttttgcgc agaaatggta aattgaagca taatagtctt | 6540 |
| gtaaggtatt tagctggctg gcgtaaagta tgctttataa aataatatat aggagtatga | 6600 |
| ttc | 6603 |

### DETAILED DESCRIPTION OF THE INVENTION

The present invention provides a probiotic comprising a GRAS microbiological organism, which GRAS microbiological organism comprises a food-grade expression vector, which vector comprises in functional linkage a nucleic acid sequence encoding for a soluble form of Amuc_1100 or a functionally equivalent fragment of said soluble form of Amuc_1100, wherein said GRAS microbiological organism is capable of expressing and secreting said soluble form of Amuc_1100 or said fragment thereof.

The term “probiotic” as used herein in the context of the present invention is defined as live microorganism, which when administered in adequate amounts, confers health benefit on the host. The probiotic may be in the form of a fermented dairy food product, a fermented non-dairy product, or a probiotic food supplement. Examples of a fermented dairy food product comprise yoghurt, yoghurt drinks, kefir, buttermilk, sour cream, viili, fil, and creme fraiche. Often dairy products are fermented are with lactic acid bacteria such as Lactococcus, Lactobacillus and Leuconostoc. However, in particular cheese may comprise bacteria and molds from other genera. Examples of fermented non-dairy products comprise pickled vegetables, sauerkraut, kimchi, pao cai, soy products including miso, tempeh, and soy sauce. Probiotic food supplements may be in the form of capsules, microcapsules, tablets, powders, and sachets, and may optionally be formulated to deliver the probiotic bacteria through the acidic environment of the stomach.

Generally recognized as safe (GRAS) is a designation of the United States Food and Drug Administration (FDA) designating that a chemical or substance added to food is considered safe by experts, and so is exempted from the usual Federal Food, Drug, and Cosmetic Act (FFDCA) food additive tolerance requirements. The term “GRAS microbiological organism” as used herein in the context of the present invention is intended to mean that the microorganism is known or is found to be suitable for consumption by a host, in particular a human, without causing a state of disease. Indeed, any organism causing a state of disease, i.e. a deterioration in health, would also not be considered as a probiotic. For example, Escherichia coli is not a GRAS microbiological organism. Thus, the terms “GRAS microbiological organism” and “probiotic” are intended to complement each other.

Microorganisms which are intended to fulfill both requirements of a “probiotic” and a “GRAS microbiological organism” are exemplified in the review article of Fijan, “Microorganisms with Claimed Probiotic Properties: An Overview of Recent Literature” Int. J. Environ. Res. Public Health 2014, 11, 4745-4765, the content of which is incorporated herein by reference. In embodiments, the GRAS microbiological organism may be selected from the group of organisms consisting of a gram-positive bacteria, a gram-negative bacteria, and a yeast. In embodiments, the GRAS microbiological organism is selected from the group consisting of organisms of the genus Lactobacillus, Bifidobacterium, Brevibacillus, Lactococcus, Enterococcus, Streptococcus, Pediococcus, Leuconostoc, Bacillus, Bacteroides, Prevotella, Parabacteroides, Ruminococcacaeae, Corynebacterium, Neisseria, Planococcaceae, Rothia, Ruminococcus, Veilonella, Coprococcus, Alistsipes, Clostridium, Lachnospiraceae, Faecalibacterium, Rikenellaceae, Comamonas, Dialister, Blautia, Roseburia, Turicibacter, and Saccharomyces. In embodiments, the GRAS microbiological organism is selected from the group consisting of organisms of the species Lactobacillus rhamnosus, Lactobacillus acidophilus, Lactobacillus plantarum, Lactobacillus casei, Lactobacillus delbrueckii subsp. bulgaricus, Lactobacillus brevies, Lactobacillus johnsonii, Lactobacillus fermentum, Lactobacillus reuteri, Bifidobacterium infantis, Bifidobacterium animalis subsp. lactis, Bifidobacterium bifidum, Bifidobacterium longum, Bifidobacterium breve, Lactococcus lactis subsp. lactis, Enterococcus durans, Enterocococcus faecium, Streptococcus thermophilus, Pediococcus acidilactici, Leuconostoc mesentoroides, Bacillus coagulans, Bacillus subtilis, Bacillus cereus, Saccharomyces boulardi. Preferably, the GRAS microbiological organism is not of the genus Akkermansia, in particular not Akkermansia muciniphila.

The invention is particularly advantageous for embodiments, wherein the GRAS microbiological organism is selected from the group of organisms consisting of a gram-positive bacteria and a gram-negative bacteria. This is because it is expected that the beneficial effects reported for Amuc_1100, in particular its Toll-like receptor 2 (TLR-2) agonistic activity, will further improve the beneficial health effects which are ascribed to the induction of TLR-2 by PAMPs found in the membrane of these microorganisms. A particular high expression of Amuc_1100 has been found in embodiments, wherein the GRAS microbiological organism is a gram-positive bacteria belonging to the order of lactic acid bacteria.

As noted above, said GRAS microbiological organism comprises a food-grade expression vector. Several food-grade expression vectors are described in the art. Food-grade expression vectors are characterized by containing only the DNA from homologous hosts or generally considered as safe organisms, and by not being dependent antibiotic markers. Consequently, said food-grade expression vector may carry a food-grade selection marker, which provides prototrophy to an otherwise auxotroph GRAS microbiological organism. Suitable vectors for lactic acid bacteria are reviewed by Landete, Critical Review in Biotechnology, 2017, 37(3): 296-308, the content of which is incorporated herein by reference. These vectors can also be used for identifying building blocks, which can be combined.

The various components of the food-grade expression vector are comprised in the vector in functional linkage. The expression “in functional linkage” as used herein, is intended to mean that the respective component of the food-grade expression vector is arranged within said vector, such that they can bring about their intended function. A marker gene is in functional linkage in case the gene is expressed such that its gene product provides the selection advantage. A replicon is in functional linkage in case the vector or plasmid is reproduced and maintained in the host cell due to the effect of said replicon. In the context of the nucleic acid encoding Amuc_1100, or a fragment thereof, said nucleic acid encoding Amuc_1100 or a fragment thereof is in functional linkage in case its gene product is expressed, such that its translated gene product is secreted into the host cells supernatant.

The food grade selection marker may be, for example, a marker selected from the group of alanine racemase (alr), thymidylate snynthase (thyA), lactose phosphotransferase (lacF), and phospho-β-galactosidase (lacG). In one particular embodiment, the marker is alanine racemase (alr), such as the alanine racemase (alr) marker encoded by SEQ ID NO: 8. The alr marker, and a food-grade expression vector using same is described in further detail in Nguyen et al., J. Agric. Food Chem. 2011, 59: 5617-5624; and Bron et al. Appl. Environ. Microbiol. 2002, 68(11): 5663-5670; each the content of which is incorporated herein by reference. In embodiments, the food-grade expression vector carries the SH71rep replicon, which has a broad functionality. The SH71rep replicon is further described by Karlskas et al., PLOS One 2014, 9(3): e91125, the content of which is incorporated herein by reference. Other suitable replicons may be employed as well. An additional 5′UTR ‘AGGAGGT’ (SEQ ID NO: 13) sequence may be optionally inserted directly upstream of the Amuc-protein sequence and 3′UTR sequence ‘TACTTGAA’ (SEQ ID NO: 14) directly downstream of the Amuc-protein sequence followed by a terminator, for example iGEM-part BBa_B1006 (SEQ ID NO: 12).

Signal sequences steering the gene of interest to the secretion pathway are known to the skilled person. For example, Dieye et al. J. Bacteriol. 2001, 183(14): 4157, the content of which is incorporated herein by reference, disclose the M6 preprotein and the Usp45 preprotein signal peptide sequence, which provides secretion when fused to the gene product of interest. Whether a gene product of interest has been expressed and secreted into the supernatant of the host cell can be tested for by assays generally known in the art, including SDS-PAGE followed by Coomassie Blue Staining, or any immunological method including dot blots, ouchterlony assays, western blots, or ELISA techniques.

In any case, the food-grade expression vector comprises in functional linkage a nucleic acid sequence encoding for a soluble form of Amuc_1100 or a fragment of said soluble form of Amuc_1100. In embodiments, the nucleic acid sequence in said food-grade expression vector encodes a soluble form of Amuc_1100 having an amino acid sequence with at least 80% identity to SEQ ID NO: 2 (Amuc_1100), such as with at least 82% identity to SEQ ID NO: 2, such as with at least 84% identity to SEQ ID NO: 2, such as with at least 86% identity to SEQ ID NO: 2, such as with at least 88% identity to SEQ ID NO: 2, such as with at least 90% identity to SEQ ID NO: 2, such as with at least 92% identity to SEQ ID NO: 2, such as with at least 94% identity to SEQ ID NO: 2, such as with at least 96% identity to SEQ ID NO: 2, such as with at least 98% identity to SEQ ID NO: 2, for example with at least 99% identity to SEQ ID NO: 2. For example, the Amuc_1100 encoded by the nucleic acid sequence comprised in functional linkage in said food-grade expression vector may comprise one or more conservative or semi-conservative substitutions, as generally known in the art, or it may be a homolog or an allelic variant to Amuc_1100 of SEQ DI NO: 2.

In one embodiment, the nucleic acid sequence in said food-grade expression vector encodes a soluble form of Amuc_1100 having an amino acid sequence as set out in SEQ ID NO: 2. A protein sequence comparison can be conducted using a sequence comparison and alignment tool, such as the publicly available program BLASTp, wherein sequence identity is intended to mean the identity of two amino acids at the same position, when both sequences are aligned, and over the total length of SEQ ID NO: 2 (287 amino acids).

In embodiments, said nucleic acid sequence may also encodes for a fragment of said soluble form of Amuc_1100, which has a length of at least 100 and up to 286 amino acids. These fragments may, for example, be N- or C-terminally truncated fragments. Alternatively, these fragments may arise from internal deletion(s). For example, said fragment may have a length of up to 285 amino acids, up to 284 amino acids, up to 283 amino acids, up to 282 amino acids, up to 281 amino acids, up to 280 amino acids, up to 275 amino acids, up to 270 amino acids, up to 265 amino acids, up to 260 amino acids, up to 255 amino acids, up to 250 amino acids, up to 240 amino acids, up to 230 amino acids, up to 220 amino acids, up to 210 amino acids, up to 200 amino acids; and/or at least 110 amino acids, at least 120 amino acids, at least 130 amino acids, at least 140 amino acids, at least 150 amino acids, at least 160 amino acids, at least 170 amino acids, at least 180 amino acids, at least 190 amino acids, at least 200 amino acids, at least 210 amino acids, at least 220 amino acids, at least 230 amino acids, at least 240 amino acids, at least 250 amino acids, at least 260 amino acids, at least 270 amino acids, or at least 280 amino acids.

In any case, the soluble Amuc_1100 protein or the fragment thereof must be selected such that it maintains at least in part the functional properties observed for Amuc_1100 of SEQ ID NO: 2. The term “functionally equivalent” or “functional properties” as used herein is intended to mean that the candidate protein maintains at least in part the property to increase the transepithelial electrical resistance (TEER), and/or the TLR-2 agonistic activity, observed for Amuc_1100 of SEQ ID NO: 2.

TLR-2 agonistic activity of the full length Amuc_1100 of SEQ ID NO: 2. TLR-2 agonistic activity can be determined using methods as described in the prior art, for example as described in Ottman et al. PLOS One 12(3): e0173004. Briefly, HEK-Blue hTLR2 cells (Invivogen, CA, USA) are grown and subcultured up to 70-80% of confluency using DMEM supplemented with 4.5 g/I D-glucose, 50 U/ml penicillin, 50 μg/ml streptomycin, 100 μg/ml Normocin, 2 mM L-glutamine, and 10% (v/v) of heat-inactivated FBS. For the experiment, cells are seeded in 180 μl in flat bottom 96-well plates and stimulated by addition of Amuc_1100 (fragment) protein to a final concentration of 5 μg/ml. Pam3CSK4 (10 ng/ml) are used as positive control, and culture medium is used as negative control. The 96-well plates are incubated for 20-24 hours at 37° C. in a 5% CO2 incubator. Stimulation of the hTLR2 receptor activates NF-κB and AP-1, which induces the production of secreted embryonic alkaline phosphatase (SEAP), the levels of which are measured spectrophotometrically. SEAP secretion is detected by measuring the OD600 at 1 hour after addition of 180 μl of QUANTI-Blue (Invivogen) to 20 μl of induced HEK-Blue hTLR2 supernatant. Experiments are performed in triplicate. The candidate soluble Amuc_1100 or the fragment thereof are considered to have or maintain TLR-2 agonistic activity in case its TLR-2 signalling activity, as determined using the foregoing assay, is at least 50% of the TLR-2 signalling activity of Amuc_1100 of SEQ ID NO: 2, such as at least 60% of the TLR-2 signalling activity of Amuc_1100 of SEQ ID NO: 2, such as at least 70% of the TLR-2 signalling activity of Amuc_1100 of SEQ ID NO: 2, such as at least 75% of the TLR-2 signalling activity of Amuc_1100 of SEQ ID NO: 2, such as at least 80% of the TLR-2 signalling activity of Amuc_1100 of SEQ ID NO: 2, for example at least 85% of the TLR-2 signalling activity of Amuc_1100 of SEQ ID NO: 2 as measured in the above-described assay.

In addition, or alternatively, the property to increase the development of transepithelial electrical resistance can be tested for using the transepithelial electrical resistance (TEER) assay, as described in Ottman et al. PLOS One 12(3): e0173004. Briefly, 5×10⁴ Caco-2 cells/insert are seeded in Millicell culture inserts with a 3 μm pore size (Merck Millipore) and grown for 8 days, whereas the growth conditions are as described in Kainulainen et al. BMC microbiology, 2015, 15(1): 4, incorporated herein by reference. Transepithelial resistance is determined using a Millicell ERS-2 TEER meter (Merck Millipore) from Caco-2 cell cultures at 0 h, and 24 h after addition of 0.5 μg/ml of Amuc_1100 protein. The candidate soluble Amuc_1100 or the fragment thereof are considered to have or maintain the property to increase the development of transepithelial electrical resistance (TEER) in case its increase in TEER compared to medium control, as determined using the foregoing assay, is at least 50% of the increase in TEER observed for Amuc_1100 of SEQ ID NO: 2, such as at least 60% of the increase in TEER observed for Amuc_1100 of SEQ ID NO: 2, such as at least 70% of the increase in TEER observed for Amuc_1100 of SEQ ID NO: 2, such as at least 75% of the increase in TEER observed for Amuc_1100 of SEQ ID NO: 2, such as at least 80% of the increase in TEER observed for Amuc_1100 of SEQ ID NO: 2, for example at least 85% of the increase in TEER observed for Amuc_1100 of SEQ ID NO: 2 as measured in the above-described assay.

Due to the degeneration of the genetic code, one and the same amino acid sequence can be encoded by different nucleic acid sequences. Indeed, different microorganisms have different preferences for encoding a particular amino acid. Depending on the abundance of the respective tRNAs in said microorganisms, expression of a gene product can be further improved by optimizing the nucleic acid sequence to the codon usage of the respective host. Thus, in embodiments, said nucleic acid sequence encoding for Amuc_1100 or a fragment thereof can be optimized for expression in a genus selected from the group of Bifidobacterium, Bacillus, Brevibacillus, Lactococcus and Saccharomyces. For example, said nucleic acid sequence may have a sequence selected from SEQ ID NO: 3 to SEQ ID NO: 7.

Within this context, said nucleic acid sequence encoding for Amuc_1100 or a fragment thereof has at least 70% sequence identity to SEQ ID NO: 1 (Amuc_1100), such as at least 72% sequence identity to SEQ ID NO: 1, such as at least 74% sequence identity to SEQ ID NO: 1, such as at least 76% sequence identity to SEQ ID NO: 1, such as at least 78% sequence identity to SEQ ID NO: 1, such as at least 80% sequence identity to SEQ ID NO: 1, such as at least 82% sequence identity to SEQ ID NO: 1, such as at least 84% sequence identity to SEQ ID NO: 1, such as at least 86% sequence identity to SEQ ID NO: 1, such as at least 88% sequence identity to SEQ ID NO: 1, such as at least 90% sequence identity to SEQ ID NO: 1, such as at least 92% sequence identity to SEQ ID NO: 1, such as at least 94% sequence identity to SEQ ID NO: 1, such as at least 96% sequence identity to SEQ ID NO: 1, such as at least 97% sequence identity to SEQ ID NO: 1, such as at least 98% sequence identity to SEQ ID NO: 1, or at least 99% sequence identity to SEQ ID NO: 1. A nucleic acid sequence comparison can be conducted using a sequence comparison and alignment tool, such as the publicly available program BLASTn, wherein sequence identity is intended to mean the identity of two nucleotides at the same position, when both sequences are aligned, and over the total length of SEQ ID NO: 1 (864 nucleotides).

In embodiments of the present invention, said soluble form of Amuc_1100 or a functionally equivalent fragment of said soluble form of Amuc_1100 does not need to comprise such a purification tag, as it is not required nor intended to purify Amuc_1100.

Moreover, while food-grade expression systems are disclosed for primary use in organisms of the genus Lactobacillus, in embodiments these expression systems are used in genera other than Lactobacillus, in which these food-grade expression vectors are also functional.

One useful example of said food-grade expression vector is p3050alrAmuc1100-sh71 (SEQ ID NO: 9) or p3050Alr_Amuc1100-sh71 with 5′UTR, 3′UTR and terminator (SEQ ID NO: 15). Many (shuttle) vectors for gram positive bacteria or for yeasts may be used, this particular vector is however the highest yielding.

In a further optional embodiment, the food-grade expression vector has an additional ethanol inducible promoter AlcA followed by human aldehyde dehydrogenase 1B1 (UniProt P30837; SEQ ID NO: 10). A corresponding food-grade expression vector is exemplified in SEQ ID NO: 11. Said vector is able to additionally express aldehyde dehydrogenase following the consumption of potable ethanol. Acetaldehyde, a metabolite of ethanol, is carcinogenic and the expression vector enables providing aldehyde dehydrogenase locally to colon, so to turn acetaldehyde into acetic acid. At the same time, it is reported that aldehyde dehydrogenase 1 expression is significantly higher in lean mice than in obese mice (Singh et al., Biochem Biophys Res Commun. 2015; 463(4): 768-773; and Yasmeen et al., Diabetes 2013; 62: 124-136; each of which is incorporated herein by reference).

Further disclosed is a method of preparing a probiotic as disclosed herein above, wherein the method comprises the step of introducing a food-grade expression vector, which vector comprises in functional linkage a nucleic acid sequence encoding for a soluble form of Amuc_1100 or a fragment of said soluble form of Amuc_1100, into a GRAS microbiological organism, such that said GRAS microbiological organism is capable of expressing and secreting said soluble form of Amuc_1100 or said fragment thereof.

Methods for introducing the vector into the GRAS microbiological organism are known in the art, and include, for example, electroporation techniques, or heat-shock techniques.

The link between gut microbiota and health is well-recognized and described, and biotherapeutic strategies evolved in the recent years, including fecal microbiota transplant (FMT), as also reviewed in Hage et al. Frontiers in Microbiology 2017, 8: article 1889, the content of which is incorporated by reference. Moreover, Plovier et al. (Nature Medicine 2016, doi: 10.1038/nm.4236, the content of which is incorporated herein by reference) and Ottman et al. (PLOS One 2017, 12(3): e0173004, the content of which is incorporated herein by reference) demonstrate that Akkermansia muciniphila or the pasteurized bacterium improve metabolism in obese and diabetic mice. It was furthermore shown that these beneficial health effects are due to a membrane protein, Amuc_1100. When added as a His-tagged purified protein in soluble form, the following beneficial health effects were observed: a reduction in body weight gain, a reduction in fat mass gain, a decrease in intestinal energy absorption, normalization of plasma LPS concentration, normalizing/reducing plasma cholesterol (in particular HDL-levels), normalizing/reducing plasma triglyceride levels, and normalizing/reducing plasma glucose levels, and improving the intestinal barrier function (as can be followed, for example, by an increase in the development of transepithelial electrical resistance).

In addition, it was demonstrated Ottman et al. (PLOS One 2017, 12(3): e0173004, the content of which is incorporated herein by reference) that the soluble, His-tagged Amuc_1100 purified protein has TLR-2 agonistic activity, and is thus considered to be involved with cross-talk with the host. In the intestine, TLR-2 regulates the expression of CYP1A1, an enzyme which is key in detoxication of certain carcinogenic substances. Recently, it was found that TLR-2 is involved in the activation of regulatory T cells (Tregs), that act to suppress immune response, thereby maintaining homeostasis and self-tolerance. It has been shown that Tregs are able to inhibit T cell proliferation and cytokine production and play a critical role in preventing autoimmunity. TLR-2 is also expressed by intestinal epithelial cells and subsets of lamina propria mononuclear cells in the gastrointestinal tract. TLR-2 has been observed downregulated in human papillomavirus-positive neoplastic keratocytres derived from uterine cervical preneoplastic lesions. Thus, TLR-2 is assumed to be associated with tumorigenesis.

Thus, in a further aspect, the above-described probiotic is for use in medicine for therapeutic purposes. Likewise, disclosed is the use of a probiotic as defined herein above for the manufacture of a medicament. Accordingly, also provided is a method of treatment of a patient, comprising the step of orally administering a probiotic as defined herein above to said patient. The patient may be a mammal, in particular a dog, cat, rat, or mouse. Preferably, the patient is a human patient. Dosages (cfu) will vary based on the formulation, the indication, and the physical state of the patient (for example dependent on the age and/or weight), but are commonly in the range of 10⁹ to 10¹⁰ CFU/day. Suitable dosages can be determined by a person skilled in the art.

More specifically, the probiotic is for use in the treatment of obesity, diabetes, and/or hypercholesterolemia. Hence, the probiotic may be used for the manufacture of a medicament for the treatment of obesity, diabetes, and/or hypercholesterolemia.

Provided is thus a method for treating obesity, diabetes, and/or hypercholesterolemia in a patient, such as a human patient, comprising the step of orally administering a probiotic as defined herein above to said patient. Similarly, also provided is a method for (i) reducing body weight gain, (ii) reducing fat mass gain, (iii) decreasing intestinal energy absorption, (iv) normalizing plasma LPS concentration, (v) normalizing/reducing plasma cholesterol (in particular HDL-levels), (vi) normalizing/reducing plasma triglyceride levels, and (vii) normalizing/reducing plasma glucose levels, and (viii) improving the intestinal barrier function in a patient, such as a human patient, comprising the step of orally administering a probiotic as defined herein above to said patient.

As used herein, the term “or” has the meaning of both “and” and “or” (i.e. “and/or”). Furthermore, the meaning of a singular noun includes that of a plural noun and thus a singular term, unless otherwise specified, may also carry the meaning of its plural form. In other words, the term “a” or “an” may mean one or more.

It is apparent to the skilled reader that, as technology develops, the basic idea of the invention can be accomplished in many different ways. The invention and its embodiments are therefore not confined to the examples described above, but may vary in the framework of patent requirements and the below claims.

### Example

If not otherwise stated, the following example uses routine methods of molecular biology, as also described in reference textbooks in the art, in particular with regard to techniques concerning molecular cloning, polymerase chain reaction, and gel electrophoresis. See, for example, ‘Molecular Cloning: A Laboratory Manual’ by Michael Green and Joseph Sambrook, 4th edition, Cold Spring Harbor Laboratory Press, Cold Spring Harbor, N.Y.

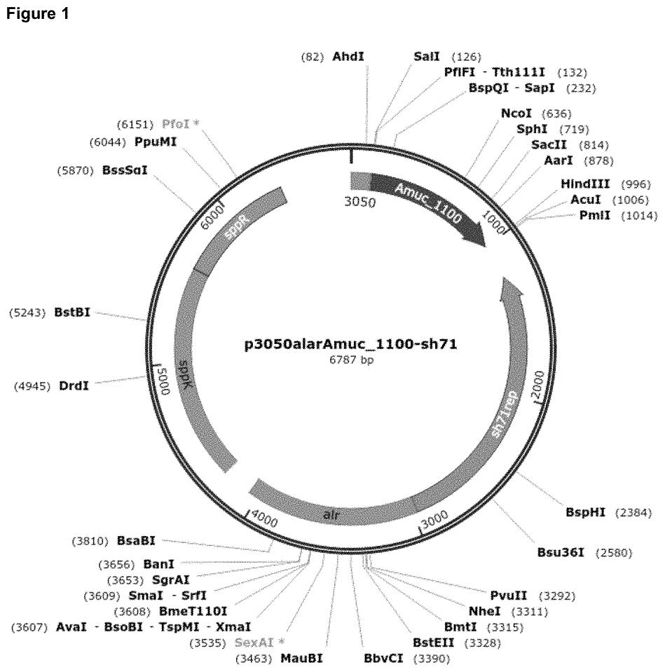

In order to construct a food-grade expression and secretion vector comprising a nucleic acid sequence encoding for a soluble form of Amuc_1100, the plasmid p3050sNucA-sh71 was selected as the starting point. The plasmid p3050sNucA-sh71 is based on pSIP411, described in Sørvig et al. Microbiology 2005, 151(7): 2439-2449 (the disclosure of which is incorporated herewith by reference), which is also the source of the sh71 replicon. The plasmid p3050sNucA-sh71 and its construction is described in Mathiesen et al. BMC Genomics 2009, 10: 425; and Karlskas et al. PLoS One, 2014, 9(3): e91125, the respective disclosure of which is hereby incorporated by reference. The plasmid p3050sNucA-sh71 (see FIG. 1 in Karlskås et al.) was first linearized by digestion with 4 restriction enzymes (BamH I, Afl III, Sal I, Hind III) yielding following bands in an agarose gel: 2852 bp (AflIII-SalI), 1962 bp (AflIII-BamHI), 1100 bp (BamHI-AflIII), 307 bp (SalI-HindIII), 178 bp (HindIII-HindIII), 17 bp (HindIII-AflIII), (linear: 2727(AflIII-End), 1962(AflIII-BamHI), 1100(BamHI-AflIII), 307(SalI-HindIII), 178(HindIII-HindIII), 125(Start-SalI), 17(HindIII-AflIII)).

The bands containing the erythromycin resistance marker gene at 1.1 kb and NucA fragments at 0.3 kb and 0.2 kb were discarded, and the DNA was cleaned.

The sh71-replicon (2 kb band) was ligated back to the backbone leaving BamHI-AflIII and SalI-HindIII restriction site pairs open to which alanine racemase and Amuc_1100 inserts were then ligated.

The food-grade alanine racemase (alr) marker gene and its isolation is described in Nguyen et al. J. Agric. Food Chem. 2011, 59, 5617-5624, the content of which is incorporated herein by reference. The following is the sequence of the alr marker gene in 5′ to 3′-direction:

alanine racemase (alr) (SEQ ID NO: 8):

| | |
|---|---|
| atgcaagcgg caactgttgt gattaaccgc cgcgctctgc gacacaacct gcaacgtctt | 60 |
| cgtgaactgg cccctgccag taaaatggtt gcggtggtga aagcgaacgc ttatggtcac | 120 |
| ggtcttcttg agaccgcgcg aacgctcccc gatgctgacg cctttggcgt agcccgtctc | 180 |
| gaagaagctc tgcgactgcg tgcgggggga atcaccaaac ctgtactgtt actcgaaggc | 240 |
| ttttttgatg ccagagatct gccgacgatt tctgcgcaac attttcatac cgccgtgcat | 300 |
| aacgaagaac agctggctgc gctggaagag gctagcctgg acgagccggt taccgtctgg | 360 |
| atgaaactcg ataccggtat gcaccgtctg ggcgtaaggc cggaacaggc tgaggcgttt | 420 |
| tatcatcgcc tgacccagtg caaaaacgtt cgtcagccgg tgaatatcgt cagccatttt | 480 |
| gcgcgcgcgg atgaaccaaa atgtggcgca accgagaaac aactcgctat ctttaatacc | 540 |
| ttttgcgaag gcaaacctgg tcaacgttcc attgccgcgt cgggtggcat tctgctgtgg | 600 |
| ccacagtcgc attttgactg ggtgcgcccg ggcatcattc tttatggcgt ctcgccgctg | 660 |
| gaagatcgct ccaccggtgc cgattttggc tgtcagccag tgatgtcact aacctccagc | 720 |
| ctgattgccg tgcgtgagca taaagccgga gagcctgttg gttatggtgg aacctgggta | 780 |
| agcgaacgtg atacccgtct tggcgtagtc gcgatgggct atggcgatgg ttatccgcgc | 840 |
| gccgcgccgt ccggtacgcc agtgctggtg aacggtcgcg aagtaccgat tgtcgggcgc | 900 |
| gtggcgatgg atatgatctg cgtagactta ggtccacagg cgcaggacaa agccggggat | 960 |
| ccggtcattt tatggggcga aggtttgccc gtagaacgta tcgctgaaat gacgaaagta | 1020 |
| agcgcttacg aacttattac gcgcctgact tcaagggtcg cgatgaaata cgtggattaa | 1080 |

For introducing same into the backbone vector, the alr selection marker was PCR-amplified with 5′ BamHI and 3′ AflIII restriction sites.

The complete nucleic acid sequence encoding for Amuc_1100 is publicly available from the KEGG GENOME Database under reference ID T00376. Isolation of Amuc_1100 from Akkermansia muciniphila is also described in Plovier et al. Nature Medicine, doi: 10.1038/nm.4236, the disclosure of which is incorporated herein by reference. The nucleic acid sequence encoding a soluble form of Amuc_1100 (i.e. an Amuc_1100 encoding gene insert lacking it's signal sequence in the N-terminal residues 1-30) was synthesized with 5′ SalI and 3′ HindIII-sites, and cloned into the above-mentioned vector backbone.

The following is the nucleic acid sequence encoding the soluble form of Amuc_1100, which lacks the first 30 N-terminal residues (in 5′ to 3′ direction): p3050Alr_Amuc1100_sh71 (SEQ ID NO: 9):

| | |
|---|---|
| atcgtcaatt ccaaacgcag tgaactggac aaaaaaatca gcatcgccgc caaggaaatc | 60 |
| aagtccgcca atgctgcgga aatcactccg agccgatcat ccaacgaaga gctggaaaaa | 120 |
| gaactgaacc gctatgccaa ggccgtgggc agcctggaaa cggcctacaa gcccttcctt | 180 |
| gcctcctccg cgctggtccc caccacgccc acggcattcc agaatgaact gaaaacattc | 240 |
| agggattccc tgatctcctc ctgcaagaaa aagaacattc tcataacgga cacatcctcc | 300 |
| tggctcggtt tccaggttta cagcacccag gctccctctg ttcaggcggc ctccacgctg | 360 |
| ggttttgaat tgaaagccat caacagcctg gtcaacaaac tggcggaatg cggcctgtcc | 420 |
| aaattcatca aggtgtaccg cccccagctc cccattgaaa ccccggcgaa caatccggaa | 480 |
| gaatcggacg aagccgacca ggccccatgg actcccatgc ctctggaaat agccttccag | 540 |
| ggcgaccggg aaagtgtatt gaaagccatg aacgccataa ccggcatgca ggactatctg | 600 |
| ttcacggtca actccatccg tatccgcaac gaacggatga tgccccctcc catcgccaat | 660 |
| ccggcagccg ccaaacctgc cgcggcccaa cccgccacgg gtgcggcttc cctgactccg | 720 |
| gcggatgagg cggctgcacc tgcagccccg gccatccagc aagtcatcaa gccttacatg | 780 |
| ggcaaggagc aggtctttgt ccaggtctcc ctgaatctgg tccacttcaa ccagcccaag | 840 |
| gctcaggaac cgtctgaaga ttaa | 864 |

The construct, p3050Alr_Amuc1100_sh71 (SEQ ID NO: 9), was then verified by DNA-sequencing and electrotransformed into the following competent probiotic strains:

### Genus

->species

### Lactobacillus

L. rhamnosus
L. acidophilus
L. plantarum
L. casei
L. delbrueckii subsp. bulgaricus
L. brevis
L. johnsonii
L. fermentum
L. reuteri

### Bifidobacterium

B. infantis
B. animalis subsp. lactis
B. bifidum
B. longum
B. breve
   Brevibacillus brevis

### Lactococcus

L. lactis subsp. lactis

### Enterococcus

E. durans
E. faecium

### Streptococcus

S. thermophilus

### Pediococcus

P. acidilactici

### Leuconostoc

L. mesentoroides

### Bacillus

B. coagulans
B. subtilis
B. cereus

### Saccharomyces

S. boulardii

Every recombinant strain secreted the protein Amuc_1100, when running the supernatant on a SDS-PAGE, and stained with Coomassie Blue.

1. A probiotic comprising a GRAS microbiological organism, which GRAS microbiological organism comprises a food-grade expression vector, which vector comprises in functional linkage a nucleic acid sequence encoding for a soluble form of Amuc_1100 or a functionally equivalent fragment of said soluble form of Amuc_1100, wherein said GRAS microbiological organism is capable of expressing and secreting said soluble form of Amuc_1100 or said fragment thereof.

2. The probiotic of claim 1, wherein the GRAS microbiological organism is selected from the group of organisms consisting of a gram-positive bacteria, a gram-negative bacteria, and a yeast.

3. The probiotic of claim 1, wherein the GRAS microbiological organism is selected from the group of organisms consisting of a gram-positive bacteria and a gram-negative bacteria.

4. The probiotic of claim 3, wherein the GRAS microbiological organism is a gram-positive bacteria of the order of lactic acid bacteria.

5. The probiotic of claim 3, wherein the GRAS microbiological organism is not of the genus Lactobacillus or of the genus Akkermansia.

6. The probiotic of claim 1, wherein the GRAS microbiological organism is selected from the group consisting of organisms of the genus Lactobacillus, Bifidobacterium, Brevibacillus, Lactococcus, Enterococcus, Streptococcus, Pediococcus, Leuconostoc, Bacillus, Bacteroides, Prevotella, Parabacteroides, Ruminococcacaeae, Corynebacterium, Neisseria, Planococcaceae, Rothia, Ruminococcus, Veilonella, Coprococcus, Alistsipes, Clostridium, Lachnospiraceae, Faecalibacterium, Rikenellaceae, Comamonas, Dialister, Blautia, Roseburia, Turicibacter, and Saccharomyces.

7. The probiotic of claim 1, wherein the GRAS microbiological organism is selected from the group consisting of organisms of the species Lactobacillus rhamnosus, Lactobacillus acidophilus, Lactobacillus plantarum, Lactobacillus casei, Lactobacillus delbrueckii subsp. bulgaricus, Lactobacillus brevies, Lactobacillus johnsonii, Lactobacillus fermentum, Lactobacillus reuteri, Bifidobacterium infantis, Bifidobacterium animalis subsp. lactis, Bifidobacterium bifidum, Bifidobacterium longum, Bifidobacterium breve, Lactococcus lactis subsp. lactis, Enterococcus durans, Enterocococcus faecium, Streptococcus thermophilus, Pediococcus acidilactici, Leuconostoc mesentoroides, Bacillus coagulans, Bacillus subtilis, Bacillus cereus, Saccharomyces boulardi.

8. The probiotic of claim 1, wherein said soluble form of Amuc_1100 or a fragment of said soluble form of Amuc_1100 does not comprise a purification tag.

9. The probiotic of claim 1, wherein said nucleic acid sequence encodes for a soluble form of Amuc_1100 having an amino acid sequence with at least 80% identity to SEQ ID NO: 2.

10. The probiotic of claim 1, wherein said nucleic acid sequence encodes for a fragment of said soluble form of Amuc_1100, which has a length of at least 100 and up to 286 amino acids.

11. The probiotic of claim 1, wherein said nucleic acid sequence is optimized for expression in the genus selected from the group of Bifidobacterium, Bacillus, Brevibacillus, Lactococcus and Saccharomyces.

12. The probiotic of claim 1, wherein said nucleic acid sequence has at least 70% identity to SEQ ID NO: 1.

13. The probiotic of claim 1, wherein said nucleic acid sequence has a sequence selected from SEQ ID NO: 3 to SEQ ID NO: 7.

14. The probiotic of claim 1, wherein said food-grade expression vector carries the SH71rep replicon.

15. The probiotic of claim 1, wherein said food-grade expression vector carries a food-grade selection marker, which provides prototrophy to the otherwise auxotroph GRAS microbiological organism.

16. The probiotic of claim 15, wherein said food grade selection marker is a marker selected from the group of alanine racemase (alr), thymidylate synthase (thyA), lactose phosphotransferase (lacF), and phospho-β-galactosidase (lacG).

17. The probiotic of claim 16, wherein said food grade selection marker is alanine racemase (alr).

18. The probiotic of claim 1, wherein said food-grade expression vector is p3050alrAmuc1100-sh71 (SEQ ID NO: 9) or p3050Alr_Amuc1100_sh71 with 5′UTR, 3′UTR and terminator (SEQ ID NO: 15).

19. The probiotic of claim 1, wherein the probiotic is in the form of a fermented non-dairy food product, a fermented dairy product, or a probiotic food supplement.

20. A method of treating a disease in a patient, comprising the step of administering orally a probiotic as defined in claim 1.

21. The method of claim 20, wherein the disease is selected from the group consisting of obesity, diabetes, hypercholesterolemia.

22. The method of claim 20, wherein the patient is a human patient.

23. A method of preparing a prebiotic according to claim 1, wherein the method comprises the step of introducing a food-grade expression vector, which vector comprises in functional linkage a nucleic acid sequence encoding for a soluble form of Amuc_1100 or a fragment of said soluble form of Amuc_1100, into a GRAS microbiological organism, such that said GRAS microbiological organism is capable of expressing and secreting said soluble form of Amuc_1100 or said fragment thereof.
